(19) United States
(12) Patent Application Publication
(10) Pub. No.: US 2025/0325697 A1
(43) Pub. Date: Oct. 23, 2025

(54) Targeting Neo Splice Sites and Cryptic Exons in the Treatment of Cancer

(71) Applicant: ST. JUDE CHILDREN'S RESEARCH HOSPITAL, INC., Memphis, TN (US)

(72) Inventors: Xiaotu MA, Memphis, TN (US); Yanling LIU, Memphis, TN (US)

(21) Appl. No.: 18/855,744

(22) PCT Filed: Mar. 30, 2023
(86) PCT No.: PCT/US2023/065129

§ 371 Date: Oct. 10, 2024

Related U.S. Application Data
(60) Provisional application No. 63/330,902, filed on Apr. 14, 2022.

(51) Int. Cl. A61K 48/00; C12N 9/22; C12N 15/11

(52) U.S. Cl. CPC A61K 48/005; C12N 9/222; C12N 15/111; C12N 2310/20

(57) ABSTRACT

Disclosed are methods and kits for eliminating cancer cells and treating cancers by targeting neo splice sites or cryptic exons of oncogenic gene fusions.

### REFERENCE TO RELATED APPLICATIONS

This application claims benefit of U.S. Provisional Patent Application Ser. No. 63/330,902, filed Apr. 14, 2022, the content of which is incorporated herein by reference in its entirety.

### STATEMENT REGARDING ELECTRONIC FILING OF A SEQUENCE LISTING

A Sequence Listing in XML format, entitled SJ0104WO_ST26.xml, 78, 050 bytes in size, generated on Mar. 24, 2023 and filed herewith, is hereby incorporated by reference into the specification for its disclosures.

### BACKGROUND

Since the discovery of Philadelphia chromosome in chronic myeloid leukemia, intensive efforts to decipher the genetic underpinnings of both adult and childhood cancers have uncovered numerous cancer driver alterations including oncogenic fusions. Longitudinal genomics studies (Ma et al. (2015) Nat. Commun. 6:6604; Li et al. (2020) Blood 135:41-55) on patient tumors under therapeutic interventions have further revealed comprehensive insights into the clonal evolution of tumors (Nowell (1976) Science 194:23-28) where cancer driving alterations can be eradicated by therapy or de novo acquired (Ma et al. (2015) Nat. Commun. 6:6604; Li et al. (2020) Blood 135:41-55). In these cases, subtype-defining oncogenic fusions (e.g., BCR-ABL1 in Philadelphia chromosome positive patients) typically remain intact during the lifetime of a tumor (Ma et al. (2015) Nat. Commun. 6:6604; Li et al. (2020) Blood 135:41-55) and can serve as stable biomarkers for determining curative outcomes. Moreover, successes in targeted inhibition of oncogenic fusions (e.g., imatinib for BCR-ABL1; Druker et al. (2001) N. Engl. J. Med. 344:1031-7) has inspired the notion of “oncogene addiction” (Weinstein (2002) Science 297:63-64) that posits on the therapeutic potential of targeting oncogenic fusions.

WO 2016/094888 A1 relates to the use of CRISPR and compositions comprising a guide RNA and a Cas protein, specifically for introducing a suicidal gene into in the breakpoint loci of a cancer-specific target sequence which is a fusion gene.

US 20201/0348161 A1 pertains to a gene-editing based cancer treatment where cancer cells are selectively eliminated by cleaving the expression product of a fusion gene or cancer inducing gene.

### SUMMARY OF THE INVENTION

This invention is a method for eliminating an oncogenic gene fusion-associated cancer cell by cleaving at least one neo splice site or cryptic exon of the gene fusion. This invention is also a method for treating a subject with an oncogenic gene fusion-associated cancer by administering an effective amount of an exogenous endonuclease that cleaves at least one neo splice site or cryptic exon of the oncogenic gene fusion of the subject. In some aspects, the oncogenic gene fusion is MN1-PATZ1, CBFB-MYH11, Cllorf95-NCOA2, TCF3-HLF, Cllorf95-MAML2, BCOR-CCNB3, EWSR1-ATF1, MN1-CXXC5, TPM3-NTRK1, SPTBN1-ALK, FUS-FLI1, KAT6A-EP300, NUP98-BPTF, EP300-BCOR, CBFA2T3-GLIS2, C11orf95-MAML2, ATXN1-NUTM2B, MRC1-PDGFRB, C11orf95-YAP1, C11orf95-RELA, NUP98-KDM5A or CIC-FOX04. In other aspects, the cleaving is done by an endonuclease selected from a CRISPR-associated protein, e.g., a Cas protein, a zinc-finger nuclease (ZFN) and a transcription activator-like effector nuclease (TALEN). In further aspects, the oncogenic gene fusion-associated cancer is a leukemia, sarcoma, lymphoma, brain cancer, liver cancer, kidney cancer, lung cancer, prostate cancer, breast cancer, ovarian cancer, colon cancer, bladder cancer, salivary gland cancer, endocrine cancer, and gastric cancer.

This invention also provides a kit including at least one endonuclease, e.g., a Cas protein, and at least one guide RNA having a targeting domain complementary to a neo splice site or cryptic exon of an oncogenic gene fusion. In particular aspects, the oncogenic gene fusion is TCF3-HLF and the at least one guide RNA is set forth in SEQ ID NO:1-7.

### BRIEF DESCRIPTION OF THE DRAWINGS

FIG. 1 shows mechanisms of oncogenic gene fusion formation. Scenario 1: the DNA breakpoints (10) can lead to fusion of coding exons (20) from N′ gene to 5′ UTR of C′ gene and result in conversion of the untranslated regions (30) into coding region, hence “neo-translational”. Scenario 2: the DNA breakpoints can lead to fusion of a coding exon from N′ gene to multiple possible coding exons of C′ gene, hence “versioning”. Scenario 3; the DNA breakpoints falling into a coding exon may disrupt the normal splice sites, and the cancer cell may use a novel splice site to ensure inclusion of corresponding exon, hence “neo-splicing”. In this scenario, a novel cryptic exon (40) may be created. Scenario 4: the DNA breakpoints may directly fuse two coding exons, hence “chimeric exon”. Scenario 5: a well-known phenomenon is promoter/enhancer hijacking.

FIG. 2 shows the study design, wherein tumor RNA sequencing data were analyzed for >5, 000 patients from multiple childhood cancer cohorts by using four different fusion detection methods, and the detected fusions were subsequently classified into versioning, neo-splicing, neo-translational, and chimeric exons.

FIG. 3 depicts an expression model for oncogenic gene fusions, wherein promoters of N′ genes are constitutionally active, while promoters of the C′ gene may or may not be constitutionally active. An expression dominance score (EDS) was used to measure the ratio of expression level (median sequencing depth) of chimeric portions between C′ gene and N′ gene for each tumor.

FIG. 4 depicts a splicing model of fusions. The oncogenic fusion defined by a DNA breakpoint (50) may or may not be subject to alternative splicing (60). A splicing dominance score (SDS) was used to measure the alternative splicing as a ratio between the count of splicing reads supporting the canonical splicing pattern (X1) divided by the count of splicing reads spanning both the N′ gene and the C′ gene (X1-X4).

FIG. 5 depicts a model of selection for fusions. DNA breakpoints from the same intron have equivalent selection pressure because they generate the same fusion proteins. DNA breakpoints from different introns may have different selection pressure when the variable exon (star) encodes critical protein domains and corresponding intron may have disproportionally more than patients other introns. A relative selection bias (RSB) score was used to measure such imbalance by accounting for patient counts (N) and intronic lengths (L) for fusion versions arrows (filled vs open arrows).

FIG. 6 shows that the neo splice donor is essential to HAL-01 by CRISPR targeting using guide g2. The induced Indels that happened to fall into coding region and lead to frameshift of TCF3-HLF are categorized into “Coding” group. Indels that directly disrupt the splice donor site are called “Loss”. Many induced Indels still leave a residual GT that may still serve as splice donor. The binding affinity of the donors after these Indels are predicted using position weight matrix (PWM) approach and categorized into different bins. Frequency of NGS reads carrying induced Indels are calculated according to such bins from day 3 to day 19 post editing. Data for three replicates is shown.

FIG. 7 depicts the pattern of neo splicing events due to incompatible exon frames between TCF3 exon 16 and HLF exon 4 in B-ALL cell line HAL-01. Guide RNAs were designed to target the cryptic exon (g₁, ATCTCAGGCGTGCCCGACTCNGG; SEQ ID NO:1) and the neo splice sites (g₂, CTGAGATTTCTGGTGCAGGINGG; SEQ ID NO:2, and gs, GATTCTATCACTCCTAGGCCNGG; SEQ ID NO:3) as well as negative control guides (g4, CTGGGGCTGGGAACTCCGTANGG; SEQ ID NO:4, bps 180 upstream of g3; g5, TGTATGACTGTATCATAACGNGG; SEQ ID NO:5, 35 bps downstream of 2). A non-template insertion sequence of 27 bp (70) was observed.

FIG. 8 depicts the pattern of neo splicing events due to incompatible exon frames between TCF3 exon 16 and HLF exon 4 in the UoC-81 cell line. Shown is the theoretic analysis of open reading frames α, β, and δ upon CRISPR editing, using either single guide or double guides. In the single guide scenario, there is always in-frame TCF3-HLF transcripts regardless of the length of induced indel. In the double guide scenario, three out of eight scenarios are predicted to disrupt all three isoforms (α, β, and δ) and lead to lethal effect.

### DETAILED DESCRIPTION OF THE INVENTION

This invention provides a therapeutic approach for eliminating cancer cells by targeting neo splice sites or the cryptic exons found in oncogenic fusion genes. Using an in vitro cell line model, the therapeutic use of CRISPR/Cas9-based genome editing of neo splicing was demonstrated and is applicable to not only neo splicing, but neo translation and cryptic exons resulting from chromosomal rearrangements in cancer cells. Advantageously, targeting of such cancer cell rearrangements with highly specific genome editing tools minimizes “on-target, off-tumor” toxicity because the method of the invention does not affect normal cells not bearing the chromosomal rearrangements.

Thus, the present invention provides a method for eliminating an oncogenic gene fusion-associated cancer cell by cleaving at least one neo splice site or cryptic exon of the oncogenic gene fusion. The term “eliminating,” “elimination,” or “eliminates” means to kill a cancer cell or otherwise diminish or reduce the number of cancer cells in a population of cells, e.g., by at least 10%, 20%, 30%, 40%, 50%, 60%, 70%, 80%, 90%, 95%, 98%, 99%, or even 100% compared to an untreated control population.

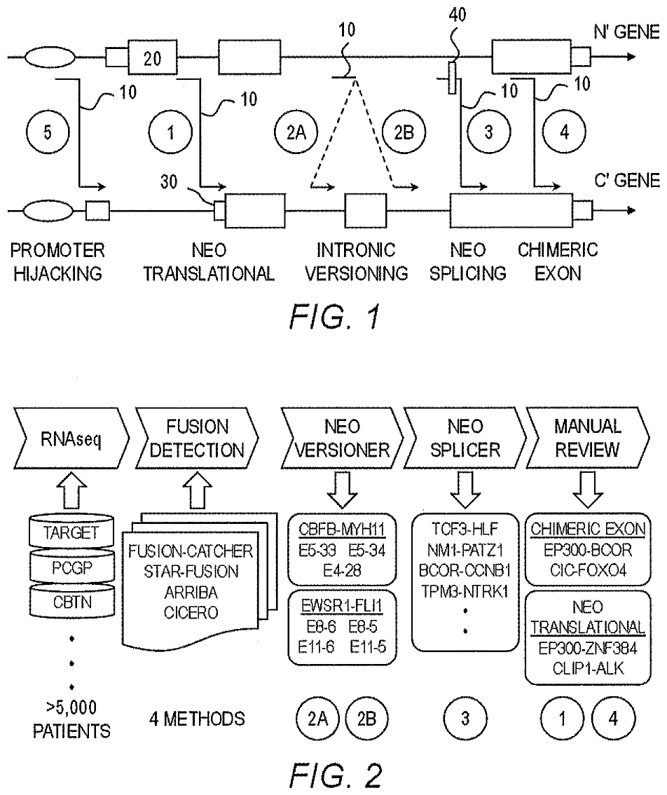

For the purposes of this invention, a neo splice site or cryptic exon is a genomic rearrangement which leads to a gene fusion that is not present in normal healthy cells (see FIG. 1). Thus, gene fusions in accordance with this invention are tumor-specific, cancer-inducing events and are therefore referred to as “oncogenic gene fusions.” In certain aspects, the gene fusion leads to the expression of a fusion product not present in normal healthy cells. Ideally, the oncogenic gene fusion/gene fusion product is critical or essential to survival of the cancer cell such that cleaving or elimination of the gene fusion/gene fusion product is lethal to the cancer cell. In this respect, the “on-target/off-tumor” toxicity is extremely very low or absent.

The term “gene fusion” or “fusion gene” as used herein means the codifying region of a gene and also, the regulatory regions and other non codifying sequences such as promoters, etc. In one aspect of this invention, the gene fusion includes at least one gene selected from MN1, PATZ1, CBFB, MYH11, Cllorf95, NCOA2, TCF3, HLF, MAML2, BCOR, CCNB3, EWSR1, ATF1, CXXC5, TPM3, NTRK1, SPTBN1, ALK, FUS, FLI1, KAT6A, EP300, NUP98, BPTE, CBFA2T3, GLIS2, ATXN1, NUTM2B, MRC1, PDGFRB, YAP1, RELA, KDM5A, CIC or FOX04. In a preferred aspect of this invention, the oncogenic gene fusion is selected from MN1-PATZ1, CBFB-MYH11, C11orf95-NCOA2, TCF3-HLF, C11orf95-MAML2, BCOR-CCNB3, EWSR1-ATF1, MN1-CXXC5, TPM3-NTRK1, SPTBN1-ALK, FUS-FLI1, KAT6A-EP300, NUP98-BPTF, EP300-BCOR, CBFA2T3-GLIS2, C11orf95-MAML2, ATXN1-NUTM2B, MRC1-PDGFRB, C11orf95-YAP1, C11orf95-RELA, NUP98-DM5A or CIC-FOX04.

As used herein, the term “cleaving”, “cleave” or “cleavage” means that one or both strands or chains of a DNA molecule (e.g., genomic DNA) are cut or one strand or chain of an RNA molecule (e.g., mRNA) is cut. Upon genome cleavage, when a double stranded molecule is cut, both sticky and blunt ends may be generated as a result of the cleavage. Ideally, cleavage of the oncogenic gene fusion in the genome leads to a deletion, an inversion, a frameshift or any combination thereof. In some aspects, cleavage does not result in the insertion of an exogenous gene, e.g., a suicide gene, as described in WO 2016/094888 A1. In some aspects, the method includes cleaving at least one, two, three, four, five or more sites of the gene fusion. Therefore, the method may include cleaving in at least one site to hundreds of sites, in cases where the genomic rearrangement includes hundreds of repetitions of a cancer-inducing oncogenic fusion gene.

In certain aspects of the invention, the cleavage is performed by at least one endonuclease. In one aspect, the endonuclease may be a CRISPR-related protein such as Cas protein, in particular a Cas9 protein, or a functional equivalent thereof, whose target site is driven by the sequence of a guide RNA. As used herein, the term “guide RNA” and “single guide RNA” are used interchangeably and are abbreviated as “gRNA” and “sgRNA.” As known in the art, ˜20 nucleotide spacer (or target domain or target sequence) of the gRNA defines the DNA or RNA target to be modified by the CRISPR-related protein. In particular, the target domain of the gRNA is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. In certain aspects of this invention, a gRNA is provided, the target domain of which is complementary to at least one neo splice site or cryptic exon of an oncogenic gene fusion.

Exemplary Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c2, C2c3, homologs thereof, or modified versions thereof. These enzymes are known, for example, the amino acid sequence of S. pyogenes Cas9 protein may be found in the SwissProt database under accession number Q99ZW2. In some aspects, the unmodified CRISPR enzyme has DNA cleavage activity, such as Cas9. In some embodiments the CRISPR enzyme is Cas9 and may be Cas9 from S. pyogenes or S. pneumoniae.

In another aspect, the cleavage is done using endonuclease Cas13. The cleavage of Cas13 of the RNA of the fusion gene is exclusive of the cancer cells and leads to the degradation of the RNA in the cell and eventually to its death. The Cas13 enzyme is a CRISPR RNA (crRNA)-guided RNA-targeting CRISPR effector. Under the guidance of a single crRNA, Cas13 can bind and cleave a target RNA carrying a complementary sequence. Through this mechanism, the CRISPR-Cas13 system can effectively knockdown mRNA expression in mammalian cells with an efficacy comparable with RNA interference technology and with improved specificity. Accordingly, in some aspects, Cas13 and crRNA are used in the methods of this invention to target a oncogenic gene fusion mRNA, in particular a cryptic exon of the mRNA.

Also, the cleaving may be performed by endonucleases such as a zinc-finger nucleases (ZFN) or transcription activator-like effector nucleases (TALEN). Both of these approaches involve applying the principles of protein-DNA interactions of these domains to engineer new proteins with unique DNA-binding specificity. These methods have been widely successful for many applications.

In a preferred aspect of the method, the cleavage is in a neo splice site of the oncogenic gene fusion. Splice sites are found at the 5′ and 3′ ends of introns. Most commonly, the RNA sequence that is removed begins with the dinucleotide GU at its 5′ end and ends with AG at its 3′ end. These consensus sequences are known to be critical, because changing one of the conserved nucleotides results in inhibition of splicing. Accordingly, in one aspect, a CRISPR-related protein such as Cas9 is used to cleave a neo splice site and the target domain of the gRNA (therefore the cleavage sequence) is specific for the neo splice site. As demonstrated herein, cleaving the genome of the cancer cells at two neo splice sites leads to the death of the cancer cell. Thus, in certain aspects, the methods of this invention provide for the use of at least two gRNA to cleave two neo splice sites. Preferred gRNAs are those codified by sequences (SEQ ID NO:1-7), useful for cleaving the TCF3-HLF fusion gene.

Another aspect of this invention provides for a kit for cleaving at least one neo splice site or cryptic exon of an oncogenic gene fusion. In one aspect, the kit includes (a) a CRISPR-associated endonuclease, preferably a Cas protein, more preferably Cas9 or a functional equivalent thereof; and (b) at least one or two gRNA to target the cleaving of the genome, preferably at a neo splice site or cryptic exon. In certain aspects, the kit includes one or more gRNAs as set forth in SEQ ID NO:1-7, which target neo splice sites of a TCF3-HLF fusion gene.

In a further aspect, the invention provides a kit including an endonuclease capable of cleaving a messenger RNA (mRNA), i.e., CRISPR associated protein Cas13 or another endonuclease derived from said Cas13 or a functional equivalent thereof (or a sequence coding said endonuclease); and at least one gRNA, i.e., crRNA, having a targeting domain specific for a cryptic exon of an oncogenic gene fusion.

Alternatively, a kit of the invention can include at least one of a zinc-finger nuclease (ZFN) or a transcription activator-like effector nuclease (TALEN), wherein said endonuclease specifically cleaves the genome at a neo splice site or cryptic exon of an oncogenic gene fusion. The kit may include the endonuclease or a sequence coding said endonuclease, preferably in an expression vector.

Another aspect of the present invention relates to the use of the methods or kits of the invention in the treatment of cancer. There are a number of cancers known in the art to be associated with or result from oncogenic gene fusions. Such cancers and their corresponding gene fusions are listed in Table 1.

TABLE 1

| | |
|---|---|
| Cancer | Oncogenic Gene Fusion |

Leukemias

| | |
|---|---|
| Acute myeloid | RUNX1-RUNX1T1, CBFB-MYH11, |
| leukemia (AML) | KMT2A-MLLT3, RPN1-MECOM, |
| | DEK-NUP214, PVT1-MECOM, |
| | RUNX1-MECOM, KMT2A-MLLT10, |
| | NUP98-NSD1, KMT2A-AFDN, |
| | CBFA2T3-GLIS2, NUP98-KDM5A, |
| | FUS-ERG, HNRNPH1-ERG, KMT2A- |
| | SEPTIN6, KAT6A-CREBBP, |
| | RUNX1-CBFA2T3 |
| Acute promyelocytic | PML-RARA, ZBTB16-RARA |
| leukemia (APL) | |
| Acute lymphocytic | ETV6-RUNX1, BCR-ABL1, TCF3- |
| leukemia (ALL) | PBX1, KMT2A-AFF1, PICALM- |
| | MLLT10, IGH-CEBPA, TCF3-HLF, |
| | TRA-MYC, KMT2A-MLLT1, KMT2A- |
| | ELL, MEF2D-BCL9, EP300- |
| | ZNF384, TCF3-ZNF384 |
| Chronic myeloid | BCR-ABL1 |
| leukemia (CML) | |
| Chronic lymphocytic | IGH-BCL1, IGH-BCL2, IGH-BCL3 |
| leukemia (CLL) | |

Sarcoma/Bone

| | |
|---|---|
| Ewing's sarcoma | EWSR1-FLI1, EWS-ERG, EWS- |
| | ETV1, EWS-FEV, EWS-E1AF |
| Alveolar | PAX3/FOXO1, PAX7-FOXO1 |
| rhabdomyosarcoma (RMS) | |
| Congenital spindle | VGLL2-CITED2, VGLL2-NCOA2, |
| cell RMS | TEAD1-NCOA2 |
| Alveolar soft-part | ASPSCR1-TFE3 |
| sarcoma | |
| Extraskeletal myxoid | EWS-TEC, TAF2N-TEC |
| chondrosarcoma | |
| Fibromyxoid sarcoma | FUS-CREB312 |
| Endometrial stromal | JAZF1-JJAZ1 |
| sarcoma | |
| Angiomatoid fibrous | EWSR1-CREB1, FUS-ATF1, |
| histiocytoma | EWSR1/ATF1 |
| Juvenile fibrosarcoma | ETV6-NTRK3 |
| Myxoid chondrosarcoma | EWS-NR4A3, TFC12-NR4A3, |
| | TAF2N-NR4A3, TAF15-NR4A3 |
| Synovial sarcoma | SYT-SSX1, SYT-SSX2, SYT-SSX4 |
| Mixoid liposarcoma | FUS-CHOP, EWS-CHOP |
| Spindle cell sarcoma | MLL4-GPS2 |
| Dermatofibrosarcoma | COL1A1-PDGFB |
| protuberans (DFSP) | |
| Clear cell sarcoma | EWS-ATF1 |
| Soft tissue angiofibroma | AHRR-NCOA2 |
| Undifferentiated round | BCOR-CCNB3, CIC-DUX4L10, |
| cell sarcoma (URCS) | CIC-DUX4 |
| Chondroid lipoma | C11ORF95-MKL2 |
| Mesenchymal | HEY1-NCOA2 |
| chondrosarcoma | |
| Biphenotypic | PAX3-M4ML3 |
| sinonasal sarcoma | |
| Despoplastic small round | EWS-WT1 |
| cell tumor | |

Lymphomas

| | |
|---|---|
| Follicular lymphoma | BCL2-IGH |
| Mantle lymphoma | BCL1-IGH |
| Large cell lymphoma | NPM-ALK |
| Burkit lymphoma | MYC-IGH |

Brain Tumors

| | |
|---|---|
| Pilocytic astrocytoma | KTAA1549-BRAF |
| Glioblastoma | TPM3-NTRK1, FGFR3-TACC3 |
| Gliomas | MYB-QKI, PPP1CB-ALK |
| Sporadic pilocytic | KIAA1549-BRAF |
| astrocytomas/some pedriatic | |
| brain tumors | |
| Supratentorial ependymomas | C11orf95-RELA, YAP1-FAM118B |
| Meningioma | MN1-ETV6 |

Liver Tumors

| | |
|---|---|
| Fibrolamellar hepatocellular | DNAJB1-PRKACA, LRIG3/ROS1 |
| carcinoma | |

Kidney Tumors

| | |
|---|---|
| Clear renal cell carcinoma | SFPQ-TFE3, TFG-GPR1228 |
| Mesoblastic nephroma | ETV6-NTRK3 |
| Renal cell carcinoma | MALAT1-TFEB |

Lung Tumors

| | |
|---|---|
| Lung adenocarcinoma | EML4-ALK, LRIG3/ROS1 |
| Non-small cell carcinoma | EML4/ALF |

Prostate Tumors

| | |
|---|---|
| Prostate cancer | TMPRSS2-ERG |

Breast/Ovarian Tumors

| | |
|---|---|
| Breast cancer | BCAS4-BCAS3, TEL1XR1-RGS17, |
| | ODZ4-NRG1 |
| Secretory breast cancer | ETV6-NTRK3 |
| Serous ovarian carcinoma | ESRRA-C11orf20 |

Colon Tumors

| | |
|---|---|
| Colorectal Cancer | PTPRK-RSPO3, TPM3-NTRK1, |
| | EIF3E-RSPO2 |

Bladder Tumors

| | |
|---|---|
| Bladder cancer | FGFR3-TACC3 |

Salivary Gland Tumors

| | |
|---|---|
| Mucoepidermoid carcinomas | MECT1-MAML2 |
| Adenoid cystic carcinoma | MYC-NFIB |
| Pleomorphic adenoma | CTNNB1-PLAG1 |

Endocrine Cancers

| | |
|---|---|
| Papillary thyroid cancer | ETV6-NTRK3 |
| (PTC) | |
| follicular thyroid cancer | PAX8-PPARG |

Endocrine Cancers

| | |
|---|---|
| Aggressive midline carcinoma | BRD4-NUT |
| Melanoma of soft parts | EWSR1-ATF1 |
| Gastric cancer | CD33-SLC1A2 |

Accordingly, the present invention also provides a method for treating a subject with an oncogenic gene fusion-associated cancer by administering an effective amount of an exogenous endonuclease that cleaves at least one neo splice site or cryptic exon of the oncogenic gene fusion of the subject. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The exogenous endonuclease can be any one of a CRISPR-associated protein, a ZEN and/or TALEN described herein. As will be understood by disclosure elsewhere herein, when using a CRISPR-associated protein such as a Cas protein, in particular a Cas9 protein, one or more gRNAs are also administered to target the CRISPR-associated protein to the target neo splice site or cryptic exon.

Cancers that can be treated in accordance with the methods of this invention include, but are not limited to, leukemias, sarcomas, lymphomas, brain cancer, liver cancer, kidney cancer, lung cancer, prostate cancer, breast cancer, ovarian cancer, colon cancer, bladder cancer, salivary gland cancer, endocrine cancer, and gastric cancer. In certain aspects, the methods of this invention are used in the treatment of a leukemia. In particular aspects, the methods of this invention are used in the treatment of ALL, AML, APL, CML or CLL. Preferably, treatment is of cancers where there is a genomic rearrangement present in a cancer cell which leads to the expression a fusion gene not present in non-cancer cells. More preferably, treatment is of the cancers listed in Table 1. Ideally, the kit of the present invention is used. In this respect, the components of the kit are delivered to the patient in need of the treatment by specific delivery systems that are known to be useful in each particular cancer type.

The terms “subject” and “patient” are used interchangeably herein. The subject treated by the present methods is desirably a human subject, although it is to be understood that the methods described herein are effective with respect to all vertebrate species, which are intended to be included in the term “subject.” Accordingly, a “subject” can include a human subject or an animal subject. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some aspects, the subject is a human including, but not limited to, fetal, neonatal, infant, juvenile, and adult subjects.

Delivery systems include conventional viral and non-viral based gene transfer methods used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome, nanoparticle or macrocomplex. Viral vector delivery systems include DNA and RNA viruses, which have either episomal or integrated genomes after delivery to the cell. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1998) Biotechnology 6 (10): 1149-1154; Vigne (1995) Restorative Neurology and Neuroscience 8:35-36; Kremer & Perricaudet (1995) British Medical Bulletin 51 (1): 31-44; Haddada et al. (1995) Current Topics in Microbiology and Immunology. Doerfler and Bohm (eds); and Yu et al. (1994) Gene Therapy 1:13-26.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in, e.g., U.S. Pat. Nos. 5,049,386, 4,946,787 and 4,897,355. Cationic and neutral lipids that are suitable for efficient receptor-recognition lipofection of polynucleotides include those described in WO 1991/17424 and WO 1991/16024. Delivery can be to cells (e.g., in vitro or ex vivo administration) or target tissues (e.g., in vivo administration).

Treatment according to the present methods can result in complete relief or cure from a cancer, or partial amelioration of one or more symptoms of the cancer, and can be temporary or permanent. The term “treatment” also is intended to encompass therapy and cure.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of the particular agent chosen, the dosing regimen to be followed, whether it is administered in combination with other compounds, timing of administration, the tissue to be imaged, and the physical delivery system in which it is carried.

The administration of kit components, i.e./endonuclease and optional gRNA, can be via different ways, depending on the target tissue or cancer cell in the patient. Thus, the administration may be oral or parenteral, subcutaneous, intramuscular or intravenous, as well as intrathecal, intracranial, etc., depending on the patient needs.

The following non-limiting examples are provided to further illustrate the present invention.

### Example 1: Materials and Methods

### Patient Cohort and RNAseq Data.

Transcriptome sequencing (RNA-seq) data from 5,286 patients were collected from following public resources: (1) St. Jude cloud (McLeod et al. (2021) Cancer Discov. 11:1082-1099) that included the St. Jude/Washington University Pediatric Cancer Genome Project cohort (PCGP; n=777; Downing et al. (2012) Nat. Genet. 44:619-622), the St. Jude Genomes for Kids study (G4K; n=253; Newman et al. (2021) Cancer Discov. 10.1158/2159-8290.CD-20-1631) and the St. Jude Real-time Clinical Genomics initiative (RTCG; n=1006); (2) a collection of transcriptome study of childhood AML (n=314); (3) a genomics study of relapsed childhood ALL (n=101; Li et al. (2020) Blood 135:41-55); (4) NCI's Therapeutically Applicable Research to Generate Effective Treatments cohort (TARGET; n=759; Ma et al. (2015) Nat. Commun. 6:6604); (5) AML transcriptome data from Children's Oncology Group (n=1086); (6) Children's Brain Tumor Network (CBTN; n=820) downloaded from Kids First data portal; and (7) Childhood Rhabdomyosarcoma (RHB; n=84; study identifier phs000720) and Ewing Sarcoma (EWS; n=84; study identifiers phs000768 and phs000804) downloaded from dbGaP. In addition, 9525 transcriptome datasets from GTEx project were used as normal controls in relevant analysis.

### Fusion Detection.

Oncogenic fusions were detected by using state-of-the-art methods reported to have superior performance (Tian et al. (2020) Genome Biol. 21:126; Haas et al. (2019) Genome Biol. 20:213), including Cicero (Tian et al. (2020) Genome Biol. 21:126), Arriba (Uhrig et al. (2021) Genome Res. 31:448-460), STAR-fusion (Haas et al. (2017) bioRxiv 120295), and FusionCatcher (Nicorici et al. (2014) bioRxiv 011650). For potential discrepancies (detected by less than two tools), the findings were manually reviewed to determine the fusion status.

Neo-Versioner. An in-house python script (“Neo-Versioner”) was developed to determine the status of intronic versioning. For each gene pair (e.g., CBFB-MYH11), the translation frame was first checked for all possible exon-exon combinations of the two involving genes to build a database of in-frame exon-exon combinations. For each in-frame exon combination, a junction contig (60 nucleotides) was next constructed using 30 nucleotides from involving exons from the N′ gene and the C′ gene, respectively. A database of 20-mers was then constructed from these contig sequences to facilitate the efficient extraction of RNAseq reads containing one of such 20-mers. Each candidate read was compared to all junction contigs. A junction contig is determined to be supported once if it is a substring of a read. To account for partial matching, a read was allowed to contain a matching of as few as 10 nucleotides from either N′ or C′ side, provided that the other side of the junction contig was fully matched to the read. The above parameters assumed an error rate of <1% in short read Illumina sequencing that is justified by recent error profile studies on next generation sequencing (Ma et al. (2019) Genome Biol. 20:50; Davis et al. (2021) Genome Biol. 22:37).

Calculating Pseudo Binding Affinity for Splice Sites The binding affinity of candidate splice site to splicing machinery was calculated using the well-established Position Specific Weight Matrix (PWM) method. Human genes were downloaded from UCSC Genome Browser, protein coding genes (RefSeq ID starts with “NM_”) and their exon boundaries were extracted and PWMs were constructed using 209, 192 donors and 205, 329 acceptors from these known protein coding genes. For donor, 3 base pairs 5′ to the GT and 10 base pairs 3′ to the GT were used, totaling a 15 base pair motif. For acceptor, 18 base pairs 5′ to the AG and 3 base pairs 3′ to the AG were used, totaling a 23 base pair motif. The motifs were denoted as Mᵢⱼ where i can be either of A, C, G or T and j=1, . . . , K where K is 15 for donor and 23 for acceptor. Mᵢⱼ represents the observed occurrences of known splice sites at position j for nucleotide i. Denote the candidate DNA sequence as Sⱼ, j=1, . . . , K, it can be scored by the PWM using a log-likelihood ratio score method:

$$LLR = {\sum }_{j} {\sum }_{i} log ( \frac{{M}_{ij}}{{B}_{i}} ) \times I ( i , {S}_{j} )$$

were Bᵢ is the genome-wide background frequency of nucleotides A, C, G and T. Here Bᵢ=0.3 when i is A or T and Bᵢ=0.2 when i is C or G to account for the A/T richness in the human genome. I(i, Sⱼ) is an indicator function that takes value of 1 when Sⱼ=i and 0 otherwise.

To ensure the quality of the constructed motifs, all splice sites of known human genes were scored and most of the splice sites received positive scores (>80% donors have score >4; >80% acceptors have score >4.3). As a negative control, 1.12 million potential donor (GT) sites and 1.76 million potential acceptor (AG) sites that do not belong to known human genes from forward strand of chr19 (one of the shortest chromosomes to save computation time) were extracted and scored. Notably, >90% of such false donors had a score <4 and >90% of such false acceptors had a score <4.3, validating the power of the PWM method in discriminating real splice sites from non-real sites.

### Neo-Splicer.

Cancer cells must create novel splice sites to allow production of functional oncogenic fusion proteins if the natural splice sites were disrupted by rearrangements. However, a novel splice may not necessarily lead to in-frame translation because multiple splice sites may be available for the cancer cells that will survive if there is one viable splicing isoform. To search for novel splice sites that can result in in-frame translation, an in-house script (“Neo-Splicer”) was developed. Given the ubiquitous nature of candidate splice sites (AG and GT; 1 in every 16 nucleotides expected by chance), the PWM described above was used to detect putative splice sites. Second, given the DNA breakpoints (42% (=834/2009) chance of detection in RNAseq data of an oncogenic fusion, all AG and GT dinucleotides were enumerated between intact exons of involving genes, hypothetical exons were generated, and corresponding translation frames were checked. RNAseq reads were then compared with above predictions to determine the neo splice sites and corresponding isoforms used by the cancer cells.

### Expression Patterns of Oncogenic Fusions.

Although N′ genes, which contributed enhancer and promoter regions for the oncogenic fusions, were expected to be constitutively expressed in the host lineage of corresponding tumor, the C′ gene may not be always expressed. An expression dominance score (EDS) was proposed to measure such expression patterns. For this, the expression level of the (fusion portion) C′ and N′ genes was first calculated as median sequencing depth (E_{c} and E_{N}) in corresponding RNAseq sample. The EDS score was then defined as EDS=E_{c}/E_{N} for each sample. For an index oncogenic fusion, the samples can be categories into (1) positive for the index fusion; (2) positive for other fusions; and (3) negative for fusions. Discrepancy in EDS scores between category (1) and categories (2) and (3) would indicate potential dysregulation of the C′ gene. Because interest was in the relative expression ratio between C′ gene and N′ gene, the global RNAseq normalization procedures (Anders et al. (2010) Genome Biol. 11:R106; Robinson et al. (2010) Bioinformatics 26:139-140) were not needed which renders EDS analysis highly efficient. Such scores are similarly calculated in non-cancer samples from GTEx cohort.

### Measuring Relative Selection Bias in Fusion Versioning.

Alternative exon (and therefore protein domain) usage due to fusion versioning can potentially lead to differential oncogenicity and therefore selection bias (although it was expected that equal oncogenicity for the different DNA breakpoints would result in a particular fusion version where the same fusion protein is produced; indeed, the nearly uniform distribution of DNA breakpoints observed indicated a lack of additional selection force when conditional on a particular fusion version). Because patient prevalence was largely predicted by gene length (more precisely, length of introns), it was posited that discrepancy between intron length and corresponding patient prevalence can predict relative selection bias (RSB). For this, the observed patient prevalence (Nᵢ) was first calculated for all versions of a given fusion. Next, the patient prevalence was normalized by the length of corresponding intron (Lᵢ). The RSB score was then defined as RBSᵢⱼ=(Nᵢ×Lⱼ)/(Nⱼ×Lᵢ), where i and j indicated the two possible introns in evaluation, in either the N′ gene or the C′ gene. A similar score can be defined for exon-exon combinations. To evaluate statistical significance, chi-square tests were performed by comparing observed patient prevalence against expected patient prevalence under the null hypothesis that involving introns carry equal selection pressure.

### Test of Uniformity of DNA Breakpoints in Intron Regions.

The uniformity of distribution of DNA breakpoints in intron regions were assessed by using a two-dimensional extension of Kolmogorov-Smirnov test that has found application in astronomy to study the clustering of stars in a pseudo 2-dimensional space.

### Splicing Dominance Score.

To measure potential alternative splicing, a splicing dominance score (SDS) was introduced. For this, the read support (Xᵢ) was first calculated for all fusion versions i (with minimum of 3 supporting reads) detected in a sample with the index fusion. Next, the dominance score was defined as SDS=X₁/ΣXᵢ. A higher SDS score would indicate lack of alternative splicing.

To study whether alternative splicing in oncogenic fusions was an inherent property of host genes, SDS scores were defined for involving genes in samples without the index fusion (wild-type) in a similar fashion. For this, the fusion-target exon of N′ gene was first defined as the most downstream exon among these fusion versions, and the fusion-target exon of C′ gene as the most upstream exon among these fusion versions. The read supports (Yᵢ) were then calculated for splicing that spanned the target exon of N′ gene (or C′ gene). The dominance score was then defined as SDS=Yᵢ/ΣYᵢ.

Samples of a matched cancer type were categorized into (1) positive for the index fusion; (2) positive for another fusion; (3) negative for all fusions to study the extent of alternative fusions and whether such property was found in corresponding wild-type genes in samples without the index fusion. This method was also applied to GTEx samples as normal control.

### Calculation of Hazard Ratio.

Event-free survival (EFS) was defined as the time since end of induction I to relapse, death, or last follow-up. Cox proportional hazard regression models were employed to estimate hazard ratios for univariable analysis of EFS in the context of fusion breakpoint and other established prognostic covariates. A p-value <0.05 was considered statistically significant.

### Cell Lines.

Cell line HAL-01 (RRID:CVCL_1242) was purchased from DSMZ, and STR profiling were performed to confirm identity, followed by whole genome and transcriptome sequencing to confirm DNA and RNA breakpoints (Table 2). STR profiling, whole genome and transcriptome sequencing were also performed to confirm identity and DNA and RNA breakpoints of the cell line UoC-B1 (RRID:CVCL_A296) (Table 2). Both cell lines are negative for mycoplasma contamination using MycoAlert Mycoplasma Detection Kit (Lonza).

TABLE 2

| | Wild-type DNA | Non-template | Wild-type DNA |
|---|---|---|---|
| Cell Line | (N′ gene; TCF3) | insertion | (C′ gene; HLF) |
| HAL-01 | GCCCTGTGCCTTCCACCA | AGGGACCGGAGT | TTTCTGGTGCAGGTGGGTC |
| | GCCCAGGAATCCTGCCTG | CGGGCACGCCTG | ATTATTTTTAACAGCTGCC |
| | CTTTCCAGGCAGACTTTC | AGA (SEQ ID | AAGTATCCCTTTGTATGAC |
| | CAAGTACCTTGATTCTAT | NO: 9) | TGTATCATAACGTGGTTGT |
| | CACTCCTAGGCCAGGGCA | | TAAATCTCCTATGCATAGT |
| | TCTCACCGCAG (SEQ | | TTTTCC |
| | ID NO: 8) | | (SEQ ID NO: 10) |
| UoC-B1 | CCCTGTGCCTTCCACCAG | TTGGTCCCCTCT | CTTGCTCACCCAGGTATTC |
| | CCCAGGAATCCTGCCTGC | CCACCTCGATCT | TTCAAAGAGCAGCCTCCTC |
| | TTTCCAGGCAGACTTTCC | A (SEQ ID | CCTCCTACCCAGAAGAATT |
| | AAGTACCTTGATTCTATC | NO: 12) | CTGGTAACATCTATTTTGA |
| | ACTCCTAGGCCAGGGCAT | | AAATCGTTTTTTTACCCTG |
| | CTCACCGCAGC (SEQ | | TTGCAT |
| | ID NO: 11) | | (SEQ ID NO: 13) |

### Cell Fitness/Dependency Assay.

One million HAL-01 or UoC-B1 cells were transiently transfected with precomplexed ribonuclear proteins (RNPs) composed of 150 pmol of chemically modified sgRNA (Synthego) and 50 pmol of SpCas9 protein (St. Jude Protein Production Core) via nucleofection (Lonza, 4D-Nucleofector™ X-unit) using solution P3 and program CA-137 in a small (20 pl) cuvette according to the manufacturer's recommended protocol. For deletion samples, a bridging ssODN donor (3 ug; IDT) was also included in the nucleofection. A portion of cells (˜10% of well) was collected at the indicated day post-nucleofection. Genomic DNA was harvested, amplified, and sequenced via deep sequencing using a 2-step library generation method. Briefly, gene-specific primers with partial Illumina adapters were used to amplify the region of interest in step 1. Gene-specific amplicons were then indexed via nested PCR using primers that bind to the partial Illumina adapters in step 2.

### NGS Analysis of Edited Cell Pools.

Upon CRISPR editing, targeted amplicon sequencing (using Illumina MiSeq) was performed on the edited cell pools to quantify the induced indels across multiple observation timepoints. For exon targeting (g₁) in cell line HAL-01, the induced indels will lead to frameshift if the length is NOT 3n (3, 6, 9 etc.), which can be analyzed by CRIS.py that measures length of target amplicon reads (Connelly & Pruett-Miller (2019) Sci. Rep. 9:4194). However, the length measurement was not suitable for analyzing splice site disruption in the edited cell pools. Therefore, dedicated in-house methods were developed to analyze such data as below.

For guide g₂ (targeting neo donor in cell line HAL-01), it was expected that the neo donor site GT would be disrupted by the induced Indel. Because it is possible that the indel can happen slightly off the desired GT dinucleotide, the algorithm was designed to account for following three possible editing scenarios: (1) the indel falls into the 5′ coding exon of desired target GT, so that it is still exon targeting per se (“coding” category); (2) the indel falls into the 3′ side of desired target GT so that it that can affect the binding affinity between splicing machinery and the donor motif; and (3) the indel directly disrupts the GT dinucleotide (“loss” category). For scenario (1), the unedited donor motif (from GT to 10 bp downstream) must be intact, and the indel must locate to the 5′ end of this motif. The translation frame of resultant mRNA was subsequently checked by assuming this donor is utilized. To account for potential decrease of binding affinity, the PWM score was also calculated for this donor motif from the mutant read. For scenario (2), the exonic sequence must be intact, and the indel must locate to the 3′ end of the exon. The PWM score was also calculated as described above. For scenario (3), neither the exonic boundary nor the unedited acceptor motif can be found in the mutant sequence. The mutant sequence is scanned for all GT dinucleotides, their PWM scores are calculated, and their translation frame status is determined by assuming they can induce splicing.

This above procedure was similarly applied for guide g₃ (targeting neo acceptor) in cell line HAL-01, except dinucleotide AG was used for acceptor and the PWM was trained from known acceptors of all human genes.

For negative controls g₄ and g₅ (that targets upstream and downstream intronic regions in HAL-1), the percentage of edited reads for 3n and non-3n indels as a negative control for guide g₁ was counted because no functional consequences were expected. Indeed, the editing rate kept ˜95% for both guides from day 3 to day 19 post editing, indicating the high efficiency of nucleofecting approach for CRISPR editing and the non-lethality of g₄ and g₅.

A similar program was written for UoC-B1 editing, although in this cell line the reading frames of all three possible exons: α, β, and δ, were simultaneously considered.

The length of CRISPR-induced indels in the data were also investigated. To account for potential sequencing errors, the analysis was limited to indels with more than 3 read support. In HAL-01, >95% of induced indels had length between −9 and 9. Therefore, “On-Target” editing was defined as indels within 10 base pairs from the designed target position, so that indels with single read support could also be included. Notably, >80% of the induced indels were insertions. For UoC-B1 double targeting, both double focal indels and single large indels were studied. Notably, double focal indels demonstrated a similar pattern to that of single guide targeting. On the other hand, the single large deletions demonstrated lengths centered around-55.

### Indel Calling.

Considering the double focal indel and large deletion in UoC-B1 experiment, a dedicated script was developed to call indels. Briefly, the wild-type DNA was first prepared as a reference sequence for this locus for BLAST program. Each NGS read was then compared against this reference. Indels were then called by maximizing the perfect match from 5′ end and then from 3′ end. All remaining DNA segments were called as “reference allele” and “mutant allele”, respectively, for the indel, along with the position. Because procedure this generated the same representation for both the large deletions and double focal indels, a post-processing step was performed to further call double focal indels. For this procedure, because the splice site between exons α and β was the critical concern, the presence of a k-mer (CCCAG|GTATT, where the vertical line is the splice site between exons α and β) was confirmed in the mutant allele of each called indel. An indel containing this k-mer was then split to call focal indels by focusing on the DNA segments to the 5′ or to the 3′ of this k-mer, respectively.

### Example 2: Model of Fusion Etiology and Study Design

Oncogenic fusions typically involve two genomic loci (genes) and said genes are denoted herein as the N′ gene (N-terminus) and C′ gene (N-terminus) for the fusion. All theoretically possible scenarios of gene fusion were enumerated (FIG. 1), where intron/exon structure and translation frame were the main constraints. This theoretical analysis revealed five fusion categories: (1) neo translational, where part of the untranslated region (5′ UTR) in C′ gene is converted into a coding region; (2) intronic versioning, where multiple introns are available to form slightly different fusion proteins; (3) neo splicing, where the DNA breakpoint disrupts natural intron/exon splicing structure so that novel splice sites are created and cryptic exons are formed; (4) chimeric exon, when DNA breakpoints fall into the coding regions of both N′ gene and C′ gene; (5) promoter/enhancer hijacking (e.g., IGH-CRLF2 in B-lineage acute lymphoblastic leukemia (B-ALL)). These five categories encompassed all possible combinations of promoter/enhancer, intron-intron, intron-exon, and exon-exon rearrangements. Because promoter/enhancer hijacking do not form a chimeric protein per se, categories (1)-(4) were focused on in this study (FIG. 2) using 5286 tumor samples from childhood cancer patients. Candidate oncogenic fusions were detected by using tools (Arriba, STAR-Fusion, Haas, Cicero, and FusionCatcher) reported to have superior performance (Tian et al. (2020) Genome Biol. 21:126; Haas et al. (2019) Genome Biol. 20:213). The detected candidate fusions were compared with previous genomics studies on childhood cancers (Ma et al. (2018) Nature 555:371-376; Roberts et al. (2014) N. Engl. J. Med. 371:1005-1015; Li et al. (2020) Blood 135:41-55; Chen et al. (2013) Cancer Cell 24:710-724; Zhang et al. (2013) Nat. Genet. 45:602-612; Crompton et al. (2014) Cancer Discov. 4:1326-1341; Parker et al. (2014) Nature 506:451-455; Shern et al. (2014) Cancer Discov. 4:216-231; Tirode et al. (2014) Cancer Discov. 4:1342-1353; Wu et al. (2014) Nat. Genet. 46:444-450; Andersson et al. (2015) Nat. Genet. 47:330-337; Lu et al. (2015) J. Invest. Dermatol. 135:816-823; Faber et al. (2016) Nat. Genet. 48:1551-1556; Bolouri et al. (2018) Nat. Med. 24:103-112; Hyrenius-Wittsten et al. (2018) Nat. Commun. 9:1770; Rusch et al. (2018) Nat. Commun. 9:3962) to establish the comprehensive list of oncogenic fusions. For detected oncogenic fusions, the fusion was classified into one of above four categories by using novel tools (Neo-Versioner and Neo-Splicer). If the fusion did not belong to either neo versioning or neo splicing categories by the automated analysis, the fusion was manually reviewed and classified into the categories of either chimeric exon or neo translational.

### Example 3: Landscape of Oncogenic Fusions in Childhood Cancers

Of the large cohort of 5,286 childhood cancer patients, oncogenic fusions were identified for 55.7% of leukemia (1, 470/2, 642), 21.7% of brain tumor (337/1, 554) and 18.7% of solid tumor (204/1, 093) patients, respectively. Among the 2, 033 oncogenic fusions, 25 neo splicing (Table 3), 24 neo translational (Table 4) and 11 chimeric exon events were detected (Table 5).

TABLE 3

| | | | |
|---|---|---|---|
| | Wild-type DNA | Non-template | Wild-type DNA |
| Fusion | (N′ gene) | insertion | (C′ gene) |

B-Cell Acute Lymphoblastic Leukemia

| | | | |
|---|---|---|---|
| TCF3_HLF | TGCCTCTTCATTCGCCTG | ACTGAGAG | ACTTGCAGTTGAGGAAA |
| | CTCCCAGACGCTGTGTGC | | TGCAGAAAATGGAAAGC |
| | CTGGCAGCGCTCAGCACT | | TGAAGTCAGCGGATCAC |
| | GGGGAAGGGGCGAGGGGT | | TACCTGTTAGAGAAAGG |
| | GCAGCAGGATGCCTCTGC | | CTTAGCCTGGCTCCCAG |
| | CTCAGGGGAGA (SEQ | | GTTGTCTTGCTTCCCA |
| | ID NO: 14) | | (SEQ ID NO: 15) |
| TCF3_HLF | GGCTGCGGGGAGGACTTG | ATTAAAA | CACTGTTGTTAACTGGA |
| | GGATTTGGCCATGAGAAA | | GGCTTCACCACTTTGGG |
| | GGTGGCAGCCGTGGAGGG | | CCCCCTCACCACCATGA |
| | CTGAGGAGGGATGGGACC | | CGTCATTGACTCGCCTG |
| | TGACCCAGGTGCTCACAG | | ACTCCTCCCAGCCTCTC |
| | ATACCCTCTGG (SEQ | | CCCTGCCTCCAGCTCC |
| | ID NO: 16) | | (SEQ ID NO: 17) |
| TCF3_HLF | GGCCCTGTGCCTTCCACC | GTCTCCAAACCC | GCTTGCTCACCCAGGTA |
| | AGCCCAGGAATCCTGCCT | (SEQ ID | TTCTTCAAAGAGCAGCC |
| | GCTTTCCAGGCAGACTTT | NO: 19) | TCCTCCCTCCTACCCAG |
| | CCAAGTACCTTGATTCTA | | AAGAATTCTGGTAACAT |
| | TCACTCCTAGGCCAGGGC | | CTATTTTGAAAATCGTT |
| | ATCTCACCGCA (SEQ | | TTTTTACCCTGTTGCA |
| | ID NO: 18) | | (SEQ ID NO: 20) |

Acute Myeloid Leukemia

| | | | |
|---|---|---|---|
| CBFB_MYH11 | TTAGAACATTATTAAAAC | | GGCCCTGTCCCTGGCTC |
| | TCGAGTAATACTACTTTC | | GGGCCCTTGAAGAGGCC |
| | CATTTTTCTATGAATATT | | TTGGAAGCCAAAGAGGA |
| | TGTCTTGGTTTTATAACT | | ACTCGAGCGGACCAACA |
| | ATAATTGTCAGTCATTTG | | AAATGCTCAAAGCCGAA |
| | TGTGATTTTAA (SEQ | | ATGGAAGACCTGGTCA |
| | ID NO: 21) | | (SEQ ID NO: 22) |
| CBFB_MYH11 | ATTACTTATTGTAACTGT | TC | GAGCTTCAGGCCGACTC |
| | ATTATTCCTAAAAGTATA | | TGCCATCAAGGGGAGGG |
| | AGGTCATGTTTAACTGAA | | AGGAAGCCATCAAGCAG |
| | ATTATATAATATTTTGAC | | CTACGCAAACTGCAGGT |
| | CCACAAATTGATTTTATT | | GGGTGACACTAGGAGCT |
| | ATATTGCTAGC (SEQ | | TGGGGCATGGGTGGAG |
| | ID NO: 23) | | (SEQ ID NO: 24) |
| CBFB_MYH11 | TATTTAGAAAAAAATAAA | | CCAAGCGGGCCCTGGAG |
| | ATTTGCTTTCAGTATTAC | | ACCCAGATGGAGGAGAT |
| | ACAGAATAATGAAAACAG | | GAAGACGCAGCTGGAAG |
| | AAGATTCTAGACCTTGCT | | AGCTGGAGGACGAGCTG |
| | ATCTCTATTCTCTGGCAT | | CAAGCCACGGAGGACGC |
| | ATAGGCTGTTT (SEQ | | CAAACTGCGGCTGGAA |
| | ID NO: 25) | | (SEQ ID NO: 26) |
| CBFB_MYH11 | TGTGATTCAGATTATCTT | | CTCAACGTGTCTACGAA |
| | TAGAGATTCAAACATCAT | | GCTGCGCCAGCTGGAGG |
| | CTAATCTAACATTATTAC | | AGGAGCGGAACAGCCTG |
| | AGTTGATAAAACTGAGAG | | CAAGACCAGCTGGACGA |
| | TCTCCAAAAAATTAATTG | | GGAGATGGAGGCCAAGC |
| | ACTTGCTCTCC (SEQ | | AGAACCTGGAGCGCCA |
| | ID NO: 27) | | (SEQ ID NO: 28) |
| FUS_FLI1 | AAAATTCCCAACTCCCAG | | GCTGGTCTTTCATTTGT |
| | CAATGCTTTGTCTGATTG | | CTTGTTTGTTTTTAAGC |
| | TTCATTTGCAGATGTCTT | | AAGAAGAATCCCTTTAG |
| | AGCGTGTTAATTTAAATG | | AGGAGGAATTAGGAAAG |
| | TCAAAGGTTTTGAGGTGT | | AAAAAAAAGTCAAACAG |
| | CCAGAACCACC (SEQ | | AAACAGAAGGAGTGGA |
| | ID NO: 29) | | (SEQ ID NO: 30) |
| KAT6A_EP300 | CCAGAATGACGACCACGA | TG | TTTTTTTTTGAGACGGA |
| | CGCTGATGATGAGGATGA | | GTTTAGCTCTTGTTGCC |
| | TGGCCACCTGGAGTCCAC | | CAGGCTGGAGTGCAGTG |
| | AAAGAAAAAGGAGCTAGA | | GTACGATCTCGGCTCAC |
| | GGAACAGCCCACGAGGGA | | TGCAACTTCTCCCTCCT |
| | AGATGTCAAGG (SEQ | | GGTTTCAAGCAACTCT |
| | ID NO: 31) | | (SEQ ID NO: 32) |
| NUP98_BPTF | AACTTTTTTGTATGGATA | | AAGTCCAAGAAAAAGAA |
| | TGTAGGGCTTGGCGAGTC | | AATGATCTCTACTACCT |
| | TAGGTCAAGCATTCCAGC | | CAAAGGAAACTAAGAAG |
| | CAAAGAATTGTGAAAGAT | | GACACAAAGCTTTACTG |
| | CACAACAATCTGGGAATA | | TATCTGTAAAACGCCTT |
| | ACAAAGATTCA (SEQ | | ATGATGAATCTAAGTG |
| | ID NO: 33) | | (SEQ ID NO: 34) |

Brain Tumor

| | | | |
|---|---|---|---|
| C11orf95_ | GGGGCGCGCTGGCCACGC | | ATCCAGAAATGTAATTT |
| NCOA2 | TCAAGGTGAGCACCATCA | | ATTCTCAGTCTTCACTG |
| | AGCGCCACATCCTGCAGG | | AAGAGCATCTGGCTCTT |
| | TGCACCCCTTCTCCATGG | | GAGCTGGAAATATGGCT |
| | ACTTCACGCCTGAGGAGC | | CTATAAGCTTTATTGTA |
| | GCCAGACTATC (SEQ | | TAGCTGAGTTTCTCTG |
| | ID NO: 35) | | (SEQ ID NO: 36) |
| Cllorf95_ | CTACCAGCCGCGGTGGCG | | TGGTATGTAAATTCAAA |
| NCOA2 | GGGCGAGTACCTGATGGA | | ACTAGAATAATAGGCTA |
| | CTACGACGGCAGCCGGCG | | CATTATGTGCTCTCATT |
| | CGGCCTGGTGTGTATGGT | | GTCTGAAAAATAAGTTC |
| | GTGCGGGGGCGCGCTGGC | | CCTGAAAAAATCCAGGA |
| | CACGCTCAAGG (SEQ | | TACCTTAAGTGATATT |
| | ID NO: 37) | | (SEQ ID NO: 38) |
| C11orf95_ | TGAGCACCATCAAGCGCC | GCGGCA | AGGTGTGGACTACCACA |
| NCOA2 | ACATCCTGCAGGTGCACC | | CCTAGCCTAAATCTAGA |
| | CCTTCTCCATGGACTTCA | | ACTTTCTATGTATATAT |
| | CGCCTGAGGAGCGCCAGA | | TTACAAATAATATTTTA |
| | CTATCCTGGAGGCCTACG | | GATTTTTGTTCTCTGGT |
| | AGGAGGCGGCG (SEQ | | TCAAATTAACTTCTCA |
| | ID NO: 39) | | (SEQ ID NO: 40) |
| C11orf95_ | CAGGTGCACCCCTTCTCC | | CGTCCGGCAACAAAGGA |
| MAML2 | ATGGACTTCACGCCTGAG | | TGTTTTGTGCTACTACT |
| | GAGCGCCAGACTATCCTG | | GAGGTTTGTGTGTGTGA |
| | GAGGCCTACGAGGAGGCG | | CTTACTTTAGAACTCTT |
| | GCGCTGCGCTGCTACGGC | | TCTAGAAAATGCGATTA |
| | CACGAGGGCTT (SEQ | | CTATTTGCATAGGTCT |
| | ID NO: 41) | | (SEQ ID NO: 42) |
| C11orf95_ | GAGCACCATCAAGCGCCA | CGGGGGGGGCGG | AAAAAATCAGAATAAAC |
| MAML2 | CATCCTGCAGGTGCACCC | CCCGAAGCCCTC | AATTTGGTCAAGTAAAA |
| | CTTCTCCATGGACTTCAC | GTGGGGTAATTA | TATTTCCCTCCAAGTAG |
| | GCCTGAGGAGCGCCAGAC | AAACGTTATTTT | TTAAGGCAAAGACTGAA |
| | TATCCTGGAGGCCTACGA | CTTTTCTTT | GGACCATTTGTAGGAAA |
| | GGAGGCGGCGC (SEQ | (SEQ ID | TGGAGAATCTTTCTAT |
| | ID NO: 43) | NO: 44) | (SEQ ID NO: 45) |
| MN1_PATZ1 | GGCAACTGAATCTAGCAG | | TCCATGCGGTCTATGTG |
| | TTTGGAGGTCTTAGAGCA | | GTAAGGTGTTCACTGAT |
| | TTTGTAATAACATGCTGG | | GCCAACCGGCTCCGGCA |
| | CTCTCTGTGAATGTCCCA | | GCACGAGGCCCAGCACG |
| | GAAGGAACATCTTCCATG | | GTGTCACCAGCCTCCAG |
| | GAATGGACTTG (SEQ | | CTGGGCTACATCGACC |
| | ID NO: 46) | | (SEQ ID NO: 47) |
| MN1_PATZ1 | ACTCTTCGTGTGTTCTTT | | GGCCTGAGGGAGGCAGG |
| | GATCAAGTCAGGACTATT | | CATCCTTCCATGCGGTC |
| | ACTTCCATTGCAGGGAGA | | TATGTGGTAAGGTGTTC |
| | CTGAGGCCCAGAGAGGGA | | ACTGATGCCAACCGGCT |
| | AAGTGCCTTGTCCAAAGT | | CCGGCAGCACGAGGCCC |
| | CACACAGCTGG (SEQ | | AGCACGGTGTCACCAG |
| | ID NO: 48) | | (SEQ ID NO: 49) |
| MN1_PATZ1 | GCTCAAGCATTGCTACGT | ACATT | CAAGCCTCTTTGCCTGT |
| | TCATTCCTTGAGATAATT | | GTTACCTGGGGTGGACC |
| | TGTGCAAAGTGGGGGAAA | | GCTTGCCCATGGTGGCT |
| | TAACCCCCTTTCAGATTT | | GGACCCCTATCCCCCCA |
| | TCTCTCTTTCTCTCTCTC | | ACTGCTGACTTCCCCAT |
| | TGTGGCAGGTA (SEQ | | TCCCCAGTGTGGCATC |
| | ID NO: 50) | | (SEQ ID NO: 51) |
| MN1_PATZ1 | CTGCTTTGCCCATCAGTC | ACGTCTCTGG | GCTGGGCTACATCGACC |
| | TGTCCTTTCAGAGTTGAA | (SEQ ID | TTCCTCCTCCGAGGCTG |
| | GCTGAGCTGCTGTTTGCT | NO: 53) | GGTGAGAATGGGCTACC |
| | GGGCAGGCCATGCAGCCC | | CATCTCTGAAGACCCCG |
| | ACACGGGGGTCCTCAGAG | | ACGGCCCCCGAAAGAGG |
| | GCCTTGCAGGG (SEQ | | AGCCGGACCAGGAAGC |
| | ID NO: 52) | | (SEQ ID NO: 54) |
| MN1_CXXC5 | CCACTGTCCTACCCGAGT | | GCGCGTGGTGCAGGAGC |
| | GAGGCTTGTTACAGACAT | | ATCTCCCGCTGATGAGC |
| | CAGGGCCCACCTGACTGT | | GAGGCGGGTGCTGGCCT |
| | GGTGGGCTACACGAGGAT | | GCCTGACATGGAGGCTG |
| | GCTCACATTTCCTCCATT | | TGGCAGGTGCCGAAGCC |
| | AGTCACCTGAT (SEQ | | CTCAATGGCCAGTCCG |
| | ID NO: 55) | | (SEQ ID NO: 56) |
| TPM3_NTRK1 | CCAGGAAGGTCTAGCTCC | | TCTCGGTGGCTGTGGGC |
| | TGACACGTTCTATGGTAG | | CTGGCCGTCTTTGCCTG |
| | AGGGAGGAGGGTTGATGC | | CCTCTTCCTTTCTACGC |
| | TTGCTCAGGTTACTTGGG | | TGCTCCTTGTGCTCAAC |
| | AACATCTCTTCCCCAGTA | | AAATGTGGACGGAGAAA |
| | TGCCTTCCAAC (SEQ | | CAAGTTTGGGATCAAC |
| | ID NO: 57) | | (SEQ ID NO: 58) |
| SPTBN1_ALK | CCGCCACTTTGCTGGCAC | | ATGCACTAGCCCACTCT |
| | CTGCTCTAAACATCTGGT | | TCCCCAAACCAGCCCTC |
| | CTCTGCTGCTTGGCTCTC | | CACCACCCTCCAGGCAG |
| | AGGAGCAAAGGTATAAGG | | AGAGATAGGAAAATCGG |
| | ACGTGGCCAATGCTAGGT | | TTTCTGAGTATATTTCT |
| | TATTAGCTTAG (SEQ | | GTTCAGCCTGTGAGCC |
| | ID NO: 59) | | (SEQ ID NO: 60) |

Solid Tumor

| | | |
|---|---|---|
| BCOR_CCNB3 | CATATGAAAATATCTCTT | CTTTTAGTAATTCAGTA |
| | CTTTATATAAGAGAAATT | CCTGTTTGAGCTAGTCT |
| | ACTCCAGTCAGAAGGACT | GTGCTTTATAGTGTGGA |
| | TAGAAACATGTTTTTTTC | GACAACTTAACTTTCCA |
| | CTTTTAAACTTTTAAGTC | GGGATTCTCAGCAGCTG |
| | AGTTTTTATGA (SEQ | ACTGGTAGCTTGCCAG |
| | ID NO: 61) | (SEQ ID NO: 62) |
| BCOR_CCNB3 | CTTGGTGATATAACTTTG | GTGTGGCCACCACACCA |
| | TTTTGTTTACAGAGTACC | GCTTTTTTTTTTTTTTT |
| | TGCTCGGGCCAGGTAAAT | TTCGTATTTTTGTAGAG |
| | GCTATTGGATGTAATCCA | ACATGGTTTCACTATGT |
| | GTAGTGTGTAATATAAAT | TGGGCAGGCTGGTCTCG |
| | TCAAACCATAT (SEQ | AACTCCTGACCTGAAG |
| | ID NO: 63) | (SEQ ID NO: 64) |
| EWSR1_ATF1 | TAAATAGCATTTTTTAAA | CAAGGTACAACTATTCT |
| | AAACAGAATGAACTTCAA | TCAGTATGCACAGACCT |
| | AATTAAAGTTGATTTTTA | CTGATGGACAGCAGATA |
| | ACTTCCATATTAGCAAAT | CTTGTGCCCAGCAATCA |
| | ACTCTTCACTACTGAAAG | GGTGGTCGTACAAAGTA |
| | ACAGTACTATT (SEQ | AGTATGCTTTCTGTCT |
| | ID NO: 65) | (SEQ ID NO: 66) |

TABLE 4

| | No. of | Gene with Neo- | Exon being neo- |
|---|---|---|---|
| Fusion | cases | translation | translated |
| PPP1CB_ALK | 1 | ALK | E1 |
| C11orf95_RELA | 2 | RELA | Intron 1; also |
| | | | chimeric |
| | | | exon/intron |
| PAX5_NCOA5 | 1 | NCOA5 | E2 |
| CLIP1_ALK | 1 | ALK | E1 |
| MAP3K8_GNG2 | 1 | GNG2 | E3 |
| TCF3_ZNF384 | 1 | ZNF384 | E3 |
| EP300_ZNF384 | 8 | ZNF384 | E3 |
| ARID1B_ZNF384 | 1 | ZNF384 | E3 |
| TAF15_ZNF384 | 2 | ZNF384 | E3 |
| SMARCA2_ZNF384 | 1 | ZNF384 | E3 |
| TCF3_ZNE384 | 3 | ZNF384 | E2 |
| EP300_ZNE384 | 2 | ZNF384 | E2 |
| MAP3K8_SVIL | 1 | SVIL | E4 |
| YAP1_FAM118B | 2 | FAM118B | E3 |
| KMT2A_MLLT11 | 3 | MLLT11 | E2 |

TABLE 5

| | Gene | | |
|---|---|---|---|
| | Orientation | | |
| Fusion | N′ | C′ | RNA contig |
| Cllorf95_YAP1 | − | + | GACGAGGAGGAGGAGCCAGAGGAGGAGGAGGAG |
| | | | GAGTGGGGCGACGTTCCGCTGTCCCCTGGAGCT |
| | | | CCCTTGGAGCGGCCCGCCGAAGAAGAGGAGGAC |
| | | | GAAGAGGACGGCCAGGAGCCTGGGGGACTCGCC |
| | | | TTGCCGCCGCCGCCTCCTCCCCCGCCTCCGCCC |
| | | | CCGCCCCGCAGCCGGGAGCAGCGGCGGAACTAC |
| | | | CAGCCGCGGTGGCGGGGCGAGTACCTGATGGAC |
| | | | TACGACGGCAGCCGGCGCGGCCTGGTGTGTATG |
| | | | GTGTGCGGGGGCGCGCTGGCCACGCTCAAGGTG |
| | | | AGCACCATCAAGCGCCACATCCTGCAGGTGCAC |
| | | | CCCTTCTCCATGGACTTCACGCCTGAGGAGCGC |
| | | | CAGACTATCCTGGAGGCCTACGAGGAGGCGGCG |
| | | | CTGCGCTGCGACCTGGAGGCGCTCTTCAACGCC |
| | | | GTCATGAACCCCAAGACGGCCAACGTGCCCCAG |
| | | | ACCGTGCCCATGAGGCTCCGGAAGCTGCCCGAC |
| | | | TCCTTCTTCAAGCCGCCGGAGCCCAAATCCCAC |
| | | | TCCCGACAGGCCAGTTGTATAGTCTCCTGTCGG |
| | | | AGACCAAAGGGTTTTGGAACTCAGAAAAAAT |
| | | | (SEQ ID NO: 67) |
| C11orf95_MAML2 | − | − | CCGGGAGCAGCGGCGGAACTACCAGCCGCGGTG |
| | | | GCGGGGCGAGTACCTGATGGACTACGACGGCAG |
| | | | CCGGCGCGGCCTGGTGTGTATGGTGTGCGGGGG |
| | | | CGCGCTGGCCACGCTCAAGGTGAGCACCATCAA |
| | | | GCGCCACATCCTGCAGGTGCACCCCTTCTCCAT |
| | | | GGACTTCACGCCTGAGGAGCGCCAGACTATCCT |
| | | | GGAGGCCTACGAGGAGGCGGCAGGAGCTGGCAA |
| | | | ACACACCAAGGCCACCGCCACTGCTGCCACCAC |
| | | | TACAGCCCCTCCACCGCCCCCTGCTGCCCCTCC |
| | | | TGCGGCCTCCCAAGCAGCAGCAACAGCAGCCCC |
| | | | ACCGCCCCCACCAGACTATCACCATCACCACCA |
| | | | GCAGCACCTGCTGAACAGTAGCAATAATGGTGG |
| | | | CAGTGGTGGGATAAACGGAGAGCAGCAGCCGCC |
| | | | CGCTTCAACCCCAGGGGACCAGAGGAACTCAGC |
| | | | CCTGATTGCGGATATTCCTTAACTGATAAGAAG |
| | | | C (SEQ ID NO: 68) |
| ATXN1_NUTM2B | − | + | GATCGACTCCAGCACCGTAGAGAGGATTGAAGA |
| | | | CAGCCATAGCCCGGGCGTGGCCGTGATACAGTT |
| | | | CGCCGTCGGGGAGCACCGAGCCCAGGTCAGCGT |
| | | | TGAAGTTTTGGTAGAGTATCCTTTTTTTGTGTT |
| | | | TGGACAGGGCTGGTCATCCTGCTGTCCGGAGAG |
| | | | AACCAGCCAGCTCTTTGATTTGCCGTGTTCCAA |
| | | | ACTCTCAGTTGGGGATGTCTGCATCTCGCTTAC |
| | | | CCTCAAGAACCTGAAGAACGGCTCTGTTAAAAA |
| | | | GGGCCAGCCCGTGGATCCCAGCAAGGCCGGCCC |
| | | | CAAGGCCCCGACTGCCTGCCTGCCACCACCCAG |
| | | | GCCCCAGAGGCCAGTGACCAAGGCCCGCCGGCC |
| | | | ACCACCCCGGCCCCACCGGCGAGCAGAGACCAA |
| | | | GGCCCGCCTGCCACCACCCAGGCCCCAGAGACC |
| | | | AGCAGAGACCAAGGTCCCTGAGGAGATCCCCCC |
| | | | AGAAGTGGTGCAGGAGTATGTGGACATCATGGA |
| | | | GGAGCTGCTAGG (SEQ ID NO: 69) |
| MRC1_PDGFRB | + | − | CCTACAAAGGATATATTTGTAAAAGACCAAAAA |
| | | | TTATTGATGCTAAACCTACTCATGAATTACTTA |
| | | | CAACAAAAGCTGACACAAGGAAGATGGACCCTT |
| | | | CTAAACCGTCTTCCAACGTGGCCGGAGTAGTCA |
| | | | TCATTGTGATCCTCCTGATTTTAACGGGTGCTG |
| | | | GCCTTGCCGCCTATTTCTTTTATAAGAAAAGAC |
| | | | GTGTGCACCTACCTCAAGAGGGCGCCTTTGAAA |
| | | | ACACTCTGTATTTTGAGTCTGTGAGCTCTGACG |
| | | | GCCATGAGTACATCTACGTGGACCCCATGCAGC |
| | | | TGCCCTATGACTCCACGTGGGAGCTGCCGCGGG |
| | | | ACCAGCTTGTGCTGGGACGCACCCTCGGCTCTG |
| | | | GGGCCTTTGGGCAGGTGGTGGAGGCCACGGCTC |
| | | | ATGGCCTGAGCCATTCTCAGGCCACGATGAAAG |
| | | | TGGCCGTCAAGATGCTTAAATCCACAGCCCGCA |
| | | | GCAGTGAGAAGCAAGCCCTTATGTCGGAGCTGA |
| | | | AGATCATGAGTCACCTTGGGCCCCAC (SEQ |
| | | | ID NO: 70) |
| EP300_BCOR | + | − | GTGCGCTCTCCCCAGCCTGTCCCTTCTCCACGG |
| | | | CCACAGTCCCAGCCCCCCCACTCCAGTCCTTCC |
| | | | CCAAGGATGCAGCCTCAGCCTTCTCCACACCAC |
| | | | GTTTCCCCACAGACAAGTTCCCCACATCCTGGA |
| | | | CTGGTAGCTGCCCAGGCCAACCCCATGGAACAA |
| | | | GGGCATTTTGCCAGCCCGGACCAGAATTCAATG |
| | | | CTTTCTCAGCTTGCTAGCAATCCAGGCATGGCA |
| | | | AACCTCCATGGTGCAAGCGCCACGGACCTGGGA |
| | | | CTCAGCACCGATTTATGTCTACCCGCTGCTTAC |
| | | | TGTGAGCGTGCAATGATGCGCTTCTCAGAGTTG |
| | | | GAGATGAAAGAAAGAGAAGGTGGCCACCCAGCA |
| | | | ACCAAAGACTCCGAGATGTGCC (SEQ ID |
| | | | NO: 71) |
| CBFAT3_GLIS2 | − | + | TGGAACTGCGGGCGGAAAGCCAGTGAGACGTGC |
| | | | AGCGGCTGCAACGCGGCACGCTACTGCGGGTCC |
| | | | TTCTGCCAGCATCGGGACTGGGAGAAGCATCAC |
| | | | CACGTGTGTGGCCAGAGCCTGCAGGGCCCCACA |
| | | | GCCGTGGTGGCCGACCCGGTGCCTGGACCGCCC |
| | | | GAAGCCGCCCACAGCCTGGGCCCCTCCCTGCCT |
| | | | GTGGGTGCTGCCAGCCTGGTGGATGACAGCCCC |
| | | | ACACCTGGCTCTCCAGGCTCCCCGCCCTCAGGC |
| | | | TTCCTGCTGAACTCCAAGTTCCCCGAGAAGGTG |
| | | | GAGGGACGCTTTTCAGCAGCCCCTCTCGTGGAC |
| | | | CTCAGCCTGTCACCACCATCTGGGCTGGACTCC |
| | | | CCCAATGGCAGCAGCTCGCTGTCCCCCGAGCGC |
| | | | CAGGGCAACGGGGACCTGCCTCCAGTG (SEQ |
| | | | ID NO: 72) |
| C11orf95_RELA | − | − | GGCGCCTGGAGAGGAGGCTGAAGGAGTCCCTGC |
| | | | AGAACTGGTTCCGGGCCGAGTGTCTCATGGACT |
| | | | ATGACCCGCGGGGGAACCGGCTGGTGTGCATGG |
| | | | CCTGTGGCCGGGCACTGCCCAGCCTGCACCTGG |
| | | | ACGACATCCGTGCCCACGTGCTGGAGGTGCACC |
| | | | CTGGCTCCCTGGGGCTCAGCGGCCCCCAGCGCA |
| | | | GTGCCCTGCTGCAGGCCTGGGGGGGCCAGCCCG |
| | | | AGGCGCTGTCTGAGCTCACCCAGTCCCCACCAG |
| | | | GCGATGACCTCGCCCCCCAGGACCTGACCGGAA |
| | | | AGAGCCGGGACTCGGCCTCCGCTGCTGGAGCCC |
| | | | CCTCCTCTCAGGATCCCTCTGGCCCCTATGTGG |
| | | | AGATCATTGAGCAGCCCAAGCAGCGGGGCATGC |
| | | | GCTTCCGCTACAAGTGCGAGGGGCGCTCCGCGG |
| | | | GCAGCATCCCAGGCGAGAGGAGCACAGATACCA |
| | | | CCAAGACCCACCCCACCATCAAGATCAATGGCT |
| | | | ACACAGGACCAGGGACAGTGCGCATCTCCCTGG |
| | | | TCACCAAGGACCCTCCTCACCGGCCTCACCCCC |
| | | | ACGAGCTTGTAGGAAAGGACTGCCGGGATGGCT |
| | | | TCTATGAGGCTGAGCTCTGCCCGGACCGCTGCA |
| | | | TCCACAGTTTCCAGAACCTGGGAATCCAGTGTG |
| | | | TGAAGAAGCGGGACCTGGAGCAGGCTATCAGTC |
| | | | AGCGCATCCAGACCAA (SEQ ID NO: 73) |
| EP300_BCOR | + | − | AAAACCTTTTGCGGACTCTCAGGTCTCCCAGCT |
| | | | CTCCCCTGCAGCAGCAACAGGTGCTTAGTATCC |
| | | | TTCACGCCAACCCCCAGCTGTTGGCTGCATTCA |
| | | | TCAAGCAGCGGGCTGCCAAGTATGCCAACTCTA |
| | | | ATCCACAACCCATCCCTGGGCAGCCTGGCATGC |
| | | | CCCAGGGGCAGCCAGGGCTACAGCCACCTACCA |
| | | | TGCCAGGTCAGCAGGGGGTCCACTCCAATCCAG |
| | | | CCATGCAGAACATGAATCCAATGCAGGCGGGCG |
| | | | TTCAGAGGGCTGGCCTGCCCCAGCAGCAACCAC |
| | | | AGCAGCAACTCCAGCCACCCATGGGAGGGATGA |
| | | | GCCCCCAGGCTCAGCAGATGAACATGAACCACA |
| | | | ACACCATGCCTTCACAATTCCGAGACATCTTGA |
| | | | GACCTGTGTTCTCCGGCTCTCCGCCCATGAAGA |
| | | | GTCTTTCATCCACCAGTGCAGGCGGCAAAAAGC |
| | | | AGGCTCAGCCAAGCTGCGCACCAGCCTCCAGGC |
| | | | CGCCTGCCAAACAGCAGAAAATTAAAGAAAACC |
| | | | AGAAGACAGATGTGCTGTGTGCAGACGAAGAAG |
| | | | AGGATTGCCAGGCTGCCTCCCTGCTGCAGAAAT |
| | | | ACACCGACAACAGCGAGAAGCCATCCGGGAAGA |
| | | | GACTGTGCAAAACCAAACACTTGATCCCTCAGG |
| | | | AGTCCAGGCGGGGATTGCCACTGACAGGGGAAT |
| | | | ACTACGTGGAGAATGCCGATGGCAAGGTGACTG |
| | | | TCCGGAGATTCAGAAAGCGGCCGGAGCCCAGTT |
| | | | CGGACTATGATCTGTCACCAGCCAAGCAGGAGC |
| | | | CAAAGCCCTTCGACCGCTTGCAGCAACTGCTAC |
| | | | CAGCCTCCCAGTCCACACAGCTGCCATGCTCAA |
| | | | GTTCCCCTCAGG (SEQ ID NO: 74) |
| CBFA2T3_GLIS2 | − | + | CGCGAGGAGCTCAACCACTGGGCGCGGCGCTAC |
| | | | AGCGACGCCGAGGACACAAAGAAGGGCCCCGCT |
| | | | CCCGCCGCGGCCCGGCCCCGCAGCAGCTCCGCC |
| | | | GGTCCCGAGTGCCTCTCGCCAGACCTGCCCCTG |
| | | | CCCAAGCAGCTGGTGTGTCGCTGGGCCAAGTGT |
| | | | AACCAGCTCT (SEQ ID NO: 75) |
| NUP98_KDM5A | − | − | ATTTGGAACAGCTCTTGGTGCTGGACAGGCATC |
| | | | TTTGTTTGGGAACAACCAACCTAAGATTGGAGG |
| | | | GCCTCTTGGTACAGGAGCCTTTGGGGCCCCTGG |
| | | | ATTTAATACTACGACAGCCACTTTGGGCTTTGG |
| | | | AGCCCCCCAGGCCCCAGTAGAAAAGGTAGAGCA |
| | | | ACTTTTTGGAGAAGGAAAACAGAAGTCCAAGGA |
| | | | GTTAAAGAAAATGGACAAACC (SEQ ID |
| | | | NO: 76) |
| CIC_FOXO4 | + | + | CTGCGGCGCACCCTGGACCAGCGCCGGGCCCTG |
| | | | GTCATGCAGCTCTTTCAGGACCATGGCTTCTTC |
| | | | CCGTCAGCCCAGGCCACAGCCGCCTTCCAGGCC |
| | | | CGCTATGCAGACATCTTTCCCTCCAAGGTTTGT |
| | | | CTGCAGTTGAAGATCCGTGAGGTGCGCCAGAAG |
| | | | ATCATGCAGGCTGCCACTCCCACGGAGCAGCCC |
| | | | CCTGGAGCTGAGGCTCCTCTCCCTGTACCGCCC |
| | | | CCCACTGGCACCGCTGCTGCCCCTGCCCCCACT |
| | | | CCCAGCCCCGCAGGGGGCCCTGACCCCACCTCA |
| | | | CCCAGCTCGGACTCTGGCACGGCCCAGGCTGCC |
| | | | CCGCCACTGCCTCCACCCCCAGAGTCGGGGCCT |
| | | | GGACAGCCTGGCTGGGAGGTTACCGGCCCCTTA |
| | | | CACACCTACAGCAGCTCCCTTTTCAGCCCAGCA |
| | | | GAGGGGCCCCTGTCAGCAGGAGAAGG (SEQ |
| | | | ID NO: 77) |

The remaining 1, 950 fusions belonged to the category of intronic versioning for leukemia (n=1, 456), brain tumor (n=319), and solid tumor (n=198) (Table 6), from which were illustrated for leukemia recurrent fusions (>5 patients), brain tumor (>3 patients) and solid tumor (>3 patients). Leukemias had the most diverse recurrent oncogenic fusions (n=26), followed by brain tumor (n=9) and solid tumor (n=6).

TABLE 6

| Cancer | Fusion |
|---|---|
| Leukemia | RUNX1-RUNX1T1, CBFB-MYH11, |
| | KMT2A-MLLT3, ETV6-RUNX1, |
| | KMT2A-MLLT10, NUP98-NSD1, |
| | BCR-ABL1, KMT2A-AFDN, |
| | TCF3-PBX1, KMT2A-AFF1, |
| | CBFA2T3-GLIS2, KMT2A-MLLT1, |
| | NUP98-KDM5A, DEK-NUP214, |
| | KMT2A-ELL, PICALM-MLLT10, |
| | FUS-ERG, MEF2D-BCL9, |
| | RBM15-MRTFA, EP300-ZNF384, |
| | HNRNPH1-ERG, KMT2A-SEPTIN6, |
| | TCF3-ZNF384, |
| | KAT6A-CREBBP, RUNX1-CBFA2T3, |
| | NIPBL-HOXB9 |
| Brain | KIAA1549-BRAF, C11orf95-RELA, |
| | FGFR1-TACC1, EWSR1-FLI1, |
| | MYB-QKI, PPP1CB-ALK, |
| | TMP3-NTRK1, YAP1-FAM118B, |
| | CLIP1-ROS1 |
| Solid | EWSR1-FLI1, PAX3-FOXO1, |
| Tumor | PAX7-FOXO1, EWSR1-ERG, |
| | EWSR1-WT1, ETV6-NTRK3 |

A high dynamic range in patient prevalence of oncogenic fusions was observed. For example, in leukemia RUNX1-RUNX1T1 was observed in 227 patients, while KMT2A-ELL was observed in 26 patients. It was hypothesized that the length of involving genes may be a contributing factor to such prevalence discrepancy. Due to the relatively smaller cohort sizes of brain tumor and solid tumor, fusions in leukemias were first analyzed. Interestingly, a marginally significant linear association (R-squared=0.23; P=0. 013) was observed between prevalence in patients and total gene length of the involved gene pairs. Considering the inherent sampling bias in the highly heterogeneous cohort from a diverse set of resources, the analysis was next limited to leukemia with rearrangement involving KMT2A, which has many known fusion partners (MLLT1, ELL, SEPTIN6, AFDN, AFF1, MLLT10, and MLLT3) with non-trivial patient prevalence (Marschalek (2016) Ann. Lab. Med. 36:85-100). Surprisingly, an excellent linear association (R-squared=0.79; P=0.008) was obtained between gene length and patient prevalence. These data indicated that among genes with oncogenic potential upon fusion, longer genes have more chance to be involved in DNA rearrangement and to generate tumors. This hypothesis implies that all eligible base pairs (under the constraints of splicing and translation frame; mostly intronic bases) in corresponding genes can contribute to functional gene fusion and therefore DNA breakpoints should be uniformly distributed along the gene. To test this hypothesis, DNA breakpoints were detected from the transcriptome data and it was found that 4 out of 5 oncogenic fusions (EWSR1-FLI1 and CBFB-MYH11) demonstrated a near-uniform distribution in their DNA breakpoints. However, an exception in TCF3-PBX1 fusion was also detected, where the DNA breakpoints tended to cluster in intron 16 of TCF3, which is consistent with previous observation (Wiemels et al. (2002) Proc. Natl. Acad. Sci. USA 99:15101-6). Together, these data indicate that random chance (or gene length), and, less frequently, local DNA properties can influence the formation of oncogenic fusions.

Extending gene length analysis to brain tumor and solid tumor did not yield statistical significance. These data may either subtypes reflect the diverse and corresponding sizes among brain tumor and solid tumor or indicate additional factors influencing the etiology of oncogenic fusions, as detailed in following sections.

### Example 4: Expression Patterns of Oncogenic Fusions

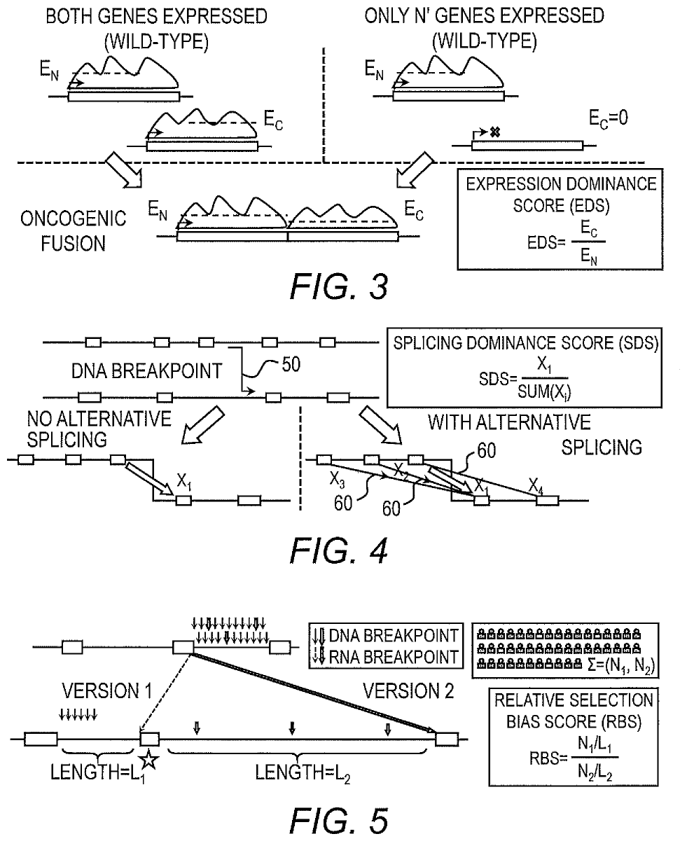

Inspired by the fusions formed by promoter/enhancer hijacking (e.g., IGH-CRLF2 or IGH-DUX4 fusion in B-ALL; Mullighan et al. (2009) Nat. Genet. 41:1243-46) that lead to aberrant activation of target genes that otherwise should be completely silenced in corresponding normal lineage of host tumor, the expression characteristics of the recurrent fusions (n≥10) was studied. This analysis was carried out by measuring the relative expression ratio between C′ gene and N′ gene using the fused portion with an expression dominance score (EDS), where a low EDS score indicated that the C′ gene was expressed at lower level than that of the N′ gene (FIG. 3). To account for the effect of gene fusion on the EDS scores, samples of a particular tumor type were categorized (so that samples with matched tissue-of-origin were used) into three groups: (1) samples with the fusion-of-interest; (2) samples with fusions other than the fusion-of-interest; (3) samples without known fusions. In the first group, the fused portion of the C′ gene must be expressed because of the fusion, while in the second and third groups, the C′ gene may or may not be expressed, and these two groups can cross validate each other. As a result, the EDS score fluctuates between-3 and 3 among samples with the fusion-of-interest (i.e., RUNX1-RUNX1T1 (E6-2), TCF3-PBX1 (E16-3), CBFA2T3-GLIS2 (E11-3), CBFB-MYH11 (E5-33), KMT2A-AFDN (E8-2), EWSR1-FLI1 (E8-6), DEK-NUP214 (E9-18), KMT2a-MLLT3 (E8-9), BCR-ABL1 (E1-2), NUP98-NSD1 (E12-7), NUP98-KDM5A (E13-27), KMT2A-ELL (E9-2), KMT2A-MLLT1 (E9-2), ETV6-RUNX1 (E5-3), KMT2A-AFF1 (E8-5), KIAA1549-BRAF (E16-9), Cllorf95-RELA (E3-2), PAX3-FOX01 (E7-2), and PAX7-FOX01 (E7-2)). On the other hand, the median EDS score in samples of group 2 and 3 can be as low as-10 in fusions including RUNX1-RUNX1T1, TCF3-PBX1, CBFB-MYH11, and CBFA2T3-GLIS2, indicating that corresponding C′ genes are typically not expressed in host lineages. For example, gene PBX1 (in fact, only the fusion portion) was expressed in B-ALL sample SJE2A059 that harbors the TCF3-PBX1 fusion but was not expressed in B-ALL sample SJBALL021772, which is TCF3-PBX1 negative. In contrast, NSD1 is constitutively expressed in AML samples both positive (SJAML064746) and negative (SJAML064774) for NUP98-NSD1 fusion. With this observation, four conventional oncogenic fusions, i.e., RUNX1-RUNX1T1, TCF3-PBX1, CBFB-MYH11, and CBFA2T3-GLIS2, with EDS scores less than-3 in group 2 and 3 samples (i.e., without the fusion of interest) were classified as “promoter hijacking-like fusions”, and the remaining fusions as conventional chimerism. Interestingly, several fusions were also observed, including KIAA1549-BRAF, Cllorf95-RELA, and PAX3/7-FOX01, to have significantly higher EDS scores in group 2 and 3 samples, indicating a highly active role of the C′ genes (BRAF, RELA, FOX01) in corresponding normal lineages. Collectively, because the C′ gene is silenced or lowly expressed in corresponding normal lineage for promoter hijacking-like fusions, it is proposed that the corresponding C′ genes (RUNX1T1, PBX1, GLIS2, and MYH11) can serve as excellent drug targets because the expected “on-target, off-tumor” toxicity can be minimized. By comparison, the “on-target, off-tumor” toxicity can be much higher in the conventional oncogenic fusion group.

### Example 5: Alternative Splicing in Oncogenic Fusions

Since alternative splicing is a general phenomenon in normal physiological conditions (Baralle & Giudice (2017) Nat. Rev. Mol. Cell Biol. 18:437-451), it was next determined whether alternative splicing can play a role in oncogenic fusions. As shown in FIG. 4, a gene fusion may or may not be subject to regulation by alternative splicing. Toward this possibility, a splicing dominance score (SDS) was designed to measure the percentage of junction reads that supports the canonical splicing (FIG. 4; open arrows) over all junction reads spanning exons in N′ gene and exons in C′ gene (FIG. 4; open and filled arrows). To determine whether alternative splicing is dependent on the rearrangement, tumor samples without the fusion of interest were also studied, wherein the SDS score was calculated as the percentage of canonical splicing over all junctions that encompass the involving intron of N′ gene and C′ genes, respectively. By applying the method to all recurrent (n>3) fusions, it was discovered that the majority (86%) of oncogenic fusions were not subject to regulation by alternative splicing. Interestingly, fusions involving KMT2A appeared to be strongly affected by alternative splicing. The detailed splicing patterns of three representative oncogenic fusions indicated that alternative usage of exon 10 in KMT2A was clearly observed in both KMT2A-rearranged AML tumors and AML tumors without KMT2A fusions. In contrast, fusion NUP98-KDM5A was not regulated by alternative splicing. On the other hand, CBFB-MYH11 appeared to have negligible (<1%) alternative splicing caused by weak exon 5 skipping that was observed in both fusion positive tumors and tumors without CBFB-MYH11. These data indicated that alternative splicing is likely a property of host gene that is not affected by somatic alterations for oncogenic fusions. To further study whether this is true in non-cancer tissues, analysis was performed in 9, 525 RNAseq samples from healthy donors in GTEx (Consortium (2013) Nat. Genet. 45:580-585). Notably, alternative splicing in ETV6-RUNX1 (identified in B-cell leukemia) was recapitulated in RUNX1 gene in normal GTEx blood samples, and alternative splicing in Cllorf95-RELA (identified in Ependymoma, a brain tumor) was recapitulated in Cllorf95 in normal GTEx brain samples. Interestingly, alternative splicing in these genes was “averaged out” when all 9,525 GTEx samples were used indiscriminately. Indeed, alternative splicing involving KMT2A was not recapitulated in GTEx dataset by this analysis, which was reflected by the lack of myeloid specimens in GTEx samples. Together, these data indicated a clear role of regulation by alternative splicing in oncogenic fusions, although such regulation is not specific to tumors and therefore is likely an intrinsic property of the host gene.

### Example 6: Selection Bias in Fusion Versioning

Because intronic versioning can cause amino acid differences in the fusion protein which may in turn lead to potential functional difference, it was hypothesized that fusion versioning could confer differential fitness to the host cells in some oncogenic fusions. It was posited that a relative selection bias score (RBS) based on the observation that DNA breakpoints are generally distributed in introns in a near-uniform fashion and gene length can predict patient prevalence. In this model, the patient prevalence of a given intron should be proportional to its length if the resultant protein versions are functionally equivalent (i.e., confers the same positive selection pressure. However, because the involved exon (FIG. 5, star) may encode functionally important protein domains and thus lead to higher positive selection pressure, its corresponding intron might have disproportionately high patient prevalence. In the cohort studied, fusion versioning was observed in 19 fusions. Here, fusions subject to alternative splicing regulation were excluded from this analysis because tumors with such fusions cannot be unambiguously classified into a particular versioning category.

A critical constraint to gene fusion products is exerted by splicing and translation, which is clearly illustrated by CBFB-MYH11 fusion in childhood AML. Here, the translational frame was first defined for each coding exon by using the codon frame of its first base. Because all six coding exons of CBFB have length of 3n=0 (mod 3), CBFB has all exons in frame 0. On the other hand, MYH11 has exon frames encompassing all three possibilities of 0, 1, and 2. Although numerous exonic combinations can theoretically generate in-frame proteins, in patients only a limited variety of fusion versions were observed, including E5-33 (n=181), E5-28 (n=16), etc. These data also indicate a potential selection bias due to critical protein domains encoded by involved exons. To test this hypothesis, a circuit plot was generated, where the N′ gene is placed on y-axis and C′ gene is placed on x-axis, and the axes are proportional to gene length. Conditional on exon 5 of CBFB, a clear discrepancy between patient prevalence and intronic length is observed for different fusion versions: intron 32 of MYH11 (corresponding to fusion version E5-33, n=181 patients) has length of only 370 bps, while intron 27 (corresponding to fusion version E5-28, n=16 patients) has a longer length of 5509 bps. With these data, a RBS score of 168.4 was observed, indicating a strong positive selection pressure for version E5-33 relative to version E5-28 (chi-square P<2×10⁻¹⁶).

To validate the hypothesis that fusion versioning may influence clinical outcomes, hazard ratios were compared for event-free survival (EFS) across the CBFB-MYH11 AML cohort (n=164) as a function of fusion versions and several well-established prognostic variables, including exon 17 KIT mutation status, white blood cell (WBC) count at diagnosis, patient age at diagnosis, and initial response to therapy as measured by end of induction I (EOI1) minimal residual disease (MRD). Remarkably, the E5-33 version of fusion CBFB-MYH11 was the best prognostic variable in this analysis, followed by exon 17 mutation KIT status, confirming that positive selection bias in version E5-33 can predict clinical outcome.

By applying this analysis to 4 fusions with recurrence >60, it was discovered that additional fusions, including ETV6-RUNX1 (Q<10⁻¹⁵) and KIAA1549-BRAF (Q=8×10⁻¹⁰) demonstrated statistically significant selection bias. On the other hand, fusion EWSR1-FLI1 only demonstrated a marginally significant Q value of 0.02 (after Bonferroni correction for multiple testing). It was noted that the limited patient number in many other fusions may have prevented the detection of selection bias. However, collectively, intronic versioning analysis provided a novel tool to study potential functional importance of certain protein domains that can serve as therapeutic targets and prognostic biomarkers.

### Example 7: Neo Splicing in Oncogenic Fusions

Oncogenic fusions harboring neo splicing were detected in 25 patient tumors (Table 3). For example, brain tumor PT_E3ADF4ZB harbored oncogenic fusion MN1-PATZ1, where the DNA breakpoint resides in exon 1 of PATZ1 and disrupts the normal splicing acceptor. To compensate for this disruption, the cancer cell created a novel splice acceptor (AG) at 26 base pairs upstream of the DNA breakpoint in intron 1 of MN1 gene. This case clearly indicated the flexibility of splicing machinery in recognizing novel splice sites. Among the oncogenic fusions with neo splicing, it was tumors with TCF3-HLF fusion discovered that all three involved neo splicing between exon 16 of TCF3 and exon 4 of HLF, indicating a common mechanism governing expression of this fusion. Indeed, close examination indicated that exon 16 of TCF3 and exon 4 of HLF have incompatible translation frames. Therefore, the neo splice sites and corresponding cryptic exons are created by the host cancer cell to compensate for the translation problem. Although it has been suggested that the cryptic exons function to make up the translation frame problem by the cancer cells (Hunger (1996) Blood 87:1211-24; Hunger et al. (1992) Genes Dev. 6:1608-20), there is no functional evidence available to date. Therefore, the function of this cryptic exon and corresponding hypothetical neo splice sites were investigated through CRISPR-based genome editing.

### Example 8: CRISPR Targeting of Neo Splicing

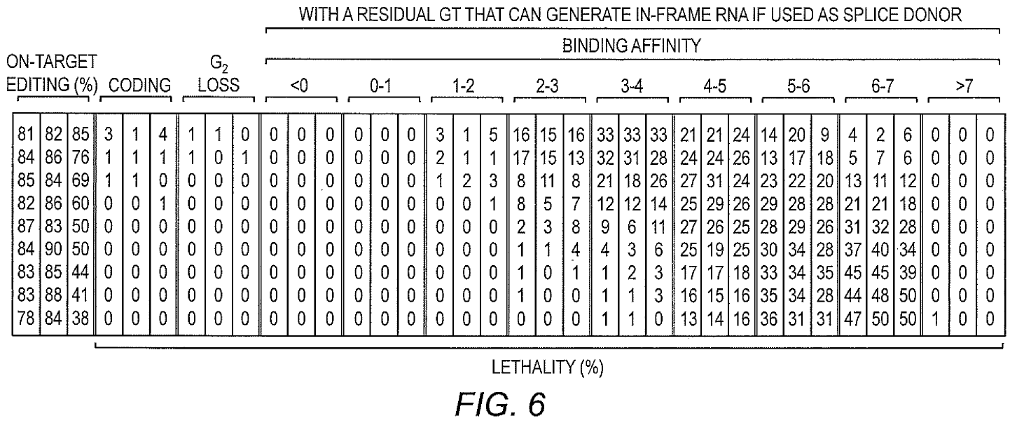

A TCF3-HLF positive cell line HAL-01 harbors a neo splicing pattern (FIG. 6) and provides an immediate in vitro model to validate the function of neo splice sites. Interestingly, this cell line harbored 27 base pairs of non-template insertion as part of the cryptic exon. Therefore, the essentiality of the cryptic exon was first tested by designing a guide RNA (g₁, ATCTCAGGCGTGCCCGACTCNGG; SEQ ID NO:1) targeting the non-template coding sequence by using the CRIPSR-Cas9 system with non-homologous end joining (NHEJ) mechanism that creates small insertion/deletions (indels). The effect of genome editing was measured using amplicon next generation sequencing (NGS) of targeted regions from day 3 through day 19 post editing. Because indels with lengths of 3n+1 and 3n+2 will cause frameshifts in this cryptic exon, it was expected that such editing would demonstrate stable reduction in abundance in NGS reads if the cryptic exon was functionally essential to the cancer cells. This analysis indicated a sharp decrease in NGS read abundance, from about 66% at day 3 to <1% by day 19 of out-of-frame indels (defined as indels with length 3n+1 and 3n+2 using CRIS.py), corresponding to >60-fold decrease with a T test P value of 0.0001. In contrast, putative non-lethal indels (defined as indels with length of 3n) demonstrated a stable increase in NGS read abundance from about 33% NGS reads at day 3 to 99% NGS reads at day 19 (Table 7). These data indicated that this cryptic exon is functionally essential for the HAL-01 cells.

TABLE 7

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 89 | 90 | 86 | 66 | 65 | 68 | 33 | 34 | 31 |
| 5 | 91 | 91 | 84 | 51 | 47 | 49 | 48 | 52 | 50 |
| 7 | 89 | 88 | 87 | 29 | 34 | 39 | 70 | 65 | 60 |
| 9 | 91 | 88 | 82 | 14 | 12 | 11 | 85 | 87 | 88 |
| 11 | 88 | 90 | 79 | 4 | 5 | 9 | 95 | 94 | 90 |
| 13 | 88 | 90 | 78 | 2 | 2 | 3 | 97 | 97 | 96 |
| 15 | 86 | 89 | 83 | 0 | 0 | 1 | 99 | 99 | 98 |
| 17 | 92 | 88 | 84 | 0 | 0 | 0 | 99 | 99 | 99 |
| 19 | 90 | 90 | 82 | 0 | 0 | 0 | 99 | 99 | 99 |

*Shown are % on-target editing (OTE; rate of induced Indels) rate, and % putative lethality (Indels causing frameshift of fusion transcripts are called lethal and other in-frame indels are called non-lethal) of NGS reads observed from day 3 to day 19 post editing for three replicates (1, 2 and 3).

The above data allowed for the investigation of the essential nature of neo splice sites in this locus. For this, a guide RNA g₂ (CTGAGATTTCTGGTGCAGGINGG; SEQ ID NO:2) was designed to target the splice donor. Because the actual (random) indel may or may not completely disrupt the splice donor, the binding affinity of residual donor site (if the GT still exists even though the indel has disrupted its context) was predicted (using a position specific weight matrix (PWM) method) and the translation frame status was simultaneously measured by assuming that such residual donor site can be used by the host cell. Only candidate donor sites with in-frame translations were evaluated for residual fitness as reflected by abundance of NGS reads from amplicon sequencing. To account for the fact that binding affinity is a continuous variable, the predicted binding affinity scores were divided into bins, and the change of NGS read abundance was studied for these score bins over time (from day 3 to day 19 post editing). Interestingly, a strong association between NGS read abundance and predicted binding affinity was observed (FIG. 6). For example, NGS reads from editing that resulted in residual donor site with binding affinity score between 2˜3 demonstrated a rapid decrease from >15% at day 3 to nearly 0% at day 19. In the next bin of binding affinity score between 3-4, NGS read abundance decreased from 33% at day 3 to ˜1% at day 19. In comparison, NGS read abundance remained at a stable 15-20% abundance when the predicted binding affinity score was 4˜5. At bin 5˜6, the NGS read abundance increased from <20% at day 3 to >30% at day 19. Strikingly, when the predicted binding affinity was in bin 6˜7, the NGS read abundance increased from ˜5% at day 3 to ˜50% at day 19, indicating a strong gain of fitness of host cells. Collectively, by using binding affinity threshold of 4, the donor editing resulted in ˜60% putative lethal on-target editing rate that was comparable to that (65%) of coding exon targeting.

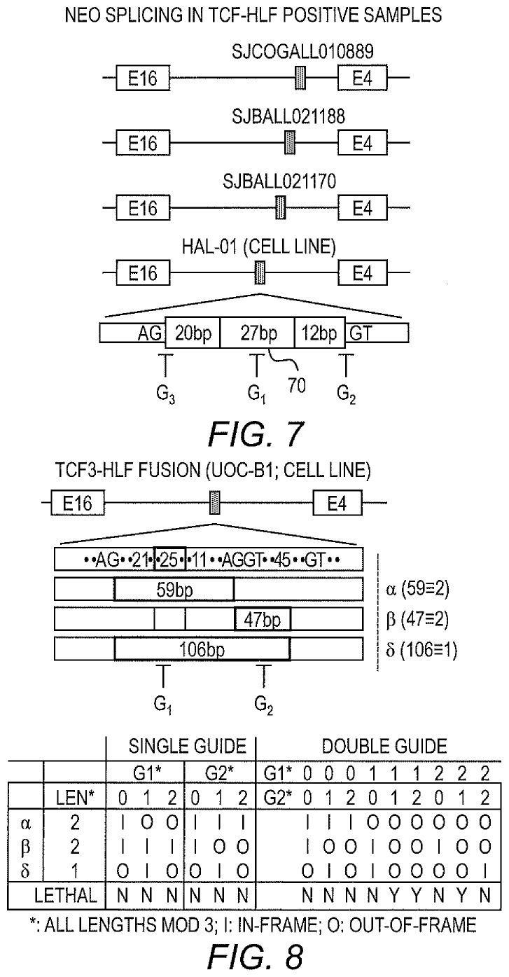

Subsequently, an attempt was made to target the neo acceptor AG by using a guide RNA g₃ (FIG. 7). The analytical procedure was similar as that of donor targeting. As it turned out, although a significant proportion (˜60%) of the editing fell into the coding region (and demonstrated expected lethal effect), ˜6% of induced indels resulted in a total loss of splice acceptor AG and demonstrated significant reduction in NGS read abundance to nearly 0% at day 19. Similar to the donor experiment, data the these clearly indicated essentiality of the neo splice acceptor which was also a therapeutic vulnerability for HAL-01 cell. Of note, targeting regions outside the cryptic exon and splice site regions had no impact on fitness, further demonstrating that the cryptic exon and its neo splice sites are a specific vulnerability to such cancer cells.

### Example 9: CRISPR Targeting in the Presence of Alternative Splicing

Although HAL-01 data indicated the feasibility of targeting the neo splice sites as well as the cryptic exon of oncogenic fusions, the potential effect of alternative splicing was not studied due to lack of natural alternative splicing in TCF3-HLF in HAL-01. For this purpose, another TCF3-HLF positive B-ALL cell line UoC-B1 was acquired, which harbors a DNA breakpoint more upstream to intron 3 of HLF than that in HAL-01, so that there are more splice site options for UoC-B1. In this line, parental UoC-B1 cells can theoretically generate three splicing isoforms by using the two candidate splicing acceptors AG and two candidate splicing donors GT (FIG. 8). Based upon published RNA sequencing for the UoC-B1 line (Accession No. SRR8816031), all three possible splicing isoforms were confirmed in parental cells: α (67% reads), β (12.5% reads), and δ (20.5% reads). Although isoforms α and β can help the UoC-B1 cells to resolve the translation frame problem between TCF3 exon 16 and HLF exon 4, isoform δ cannot. Therefore, it was predicted that targeting isoforms α or β alone may not be effective due to compensatory splicing among them. To test this hypothesis, one guide (g₁, CAAGTAGATCGAGGTGGAGANGG; SEQ ID NO:6) was designed to target isoform & and another guide (g₂, CCAGAATTCTTCTGGGTAGGNGG; SEQ ID NO:7) to target isoform β. This analysis indicated that g₁ and g₂ alone lead to negligible reduction of putative “lethal” on-target editing that disrupted α and β, respectively (g₁, Table 8; g₂, Table 9). These data confirm the compensatory role of α and β exons when perturbed alone.

TABLE 8

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 82 | 85 | 85 | 64 | 65 | 66 | 20 | 18 | 20 |
| 5 | 82 | 84 | 85 | 64 | 58 | 61 | 23 | 25 | 23 |
| 7 | 83 | 84 | 86 | 60 | 58 | 59 | 31 | 23 | 25 |
| 9 | 85 | 87 | 84 | 59 | 53 | 55 | 31 | 27 | 29 |
| 11 | 85 | 79 | 87 | 57 | 53 | 63 | 33 | 27 | 25 |
| 13 | 85 | 87 | 84 | 55 | 51 | 60 | 29 | 24 | 23 |
| 15 | 84 | 83 | 85 | 56 | 50 | 60 | 30 | 24 | 24 |
| 17 | 87 | 86 | 81 | 54 | 50 | 54 | 30 | 23 | 25 |
| 19 | 85 | 84 | 84 | 54 | 49 | 56 | 31 | 24 | 23 |

*Shown are OTE and % putative lethality of NGS reads observed from day 3 to day 19 post editing for three replicates (1, 2 and 3).

TABLE 9

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 79 | 77 | 81 | 42 | 45 | 39 | 18 | 18 | 23 |
| 5 | 85 | 83 | 83 | 47 | 44 | 42 | 18 | 19 | 18 |
| 7 | 84 | 84 | 84 | 43 | 44 | 40 | 18 | 20 | 21 |
| 9 | 86 | 85 | 84 | 41 | 39 | 37 | 20 | 20 | 20 |
| 11 | 85 | 86 | ND | 39 | 35 | ND | 18 | 20 | ND |
| 13 | 85 | 87 | 84 | 32 | 29 | 30 | 19 | 21 | 19 |
| 15 | 84 | 86 | 87 | 32 | 29 | 27 | 18 | 22 | 18 |
| 17 | 85 | 86 | 85 | 32 | 32 | 26 | 18 | 20 | 21 |
| 19 | 86 | 87 | 85 | 27 | 27 | 25 | 20 | 18 | 23 |

*Shown are OTE and % putative lethality of NGS reads observed from day 3 to day 19 post editing for three replicates (1, 2 and 3). ND, no data.

It was posited that double editing that simultaneously disrupts all possible isoforms may lead t synthetic lethality. For this, the theoretical possibilities of CRISPR targeting were analyzed using double guides g₁+g₂. By categorizing the effect of induced indels into two states (being in-frame (I) or being out-of-frame (O)) for each of the three isoforms α, β, and δ, it was predicted that only reads that lead to “O” state for all three isoforms can result in lethal effect, which comprises 37.5% (=3/8) of all on-target editing. This analysis (g₁+g₂, Table 10) indicated that the putative lethal editing demonstrated a sharp decrease of NGS read abundance from ˜37% at day 3 to nearly 0% at day 19.

TABLE 10

| | OTE | | | % Lethal | | | % Non-Lethal | | |
|---|---|---|---|---|---|---|---|---|---|
| Day | 1 | 2 | 3 | 1 | 2 | 3 | 1 | 2 | 3 |
| 3 | 78 | 83 | 83 | 40 | 35 | 39 | 19 | 20 | 22 |
| 5 | 83 | 84 | 81 | 31 | 35 | 30 | 22 | 22 | 25 |
| 7 | 85 | 83 | 83 | 18 | 17 | 13 | 29 | 22 | 25 |
| 9 | 81 | 86 | 85 | 7 | 6 | 6 | 26 | 20 | 23 |
| 11 | 84 | 84 | 84 | 5 | 5 | 6 | 26 | 21 | 25 |
| 13 | 83 | 85 | 83 | 4 | 2 | 1 | 22 | 16 | 18 |
| 15 | 85 | 86 | 85 | 2 | 0 | 0 | 23 | 15 | 20 |
| 17 | 86 | 86 | 85 | 1 | 0 | 1 | 20 | 13 | 15 |
| 19 | 83 | 85 | 86 | 0 | 0 | 0 | 23 | 13 | 19 |

*Shown are OTE and % putative lethality of NGS reads observed from day 3 to day 19 post editing for three replicates (1, 2 and 3).

In contrast, the putative non-lethal editing (that can keep at least one of α, β, and δ being in-frame) remained a stable NGS read abundance from day 3 to day 19. Because double guides theoretically can lead to double focal indel editing and single large deletion, the NGS reads of these two categories were also studied. Indeed, nearly 50% of lethal editing are large deletions, and both large deletions and double focal indels had comparable decreases in NGS read abundance. These data clearly demonstrated the functionally compensatory nature of alternative splicing in TCF3-HLF in UoC-B1 that posed a significant challenge in gene targeting using only single guide approach.

Together, these experiments indicated that neo splicing in corresponding oncogenic fusions are functionally essential for host cancer cells and offer novel therapeutic vulnerability. To facilitate targeting, computational approaches are used to accurately predict outcomes of CRISPR editing to enable rationale design of CRISPR guides and minimize escaping effect.

What is claimed is:

1. A method for eliminating an oncogenic gene fusion-associated cancer cell comprising cleaving at least one neo splice site or cryptic exon of the gene fusion thereby eliminating the oncogenic gene fusion-associated cancer cell.

2. The method of claim 1, wherein the oncogenic gene fusion-associated cancer cell is a leukemia cell.

3. The method of claim 2, wherein the oncogenic gene fusion is MN1-PATZ1, CBFB-MYH11, C11orf95-NCOA2, TCF3-HLF, C11orf95-MAML2, BCOR-CCNB3, EWSR1-ATF1, MN1-CXXC5, TPM3-NTRK1, SPTBN1-ALK, FUS-FLI1, KAT6A-EP300, NUP98-BPTF, EP300-BCOR, CBFA2T3-GLIS2, C11orf95-MAML2, ATXN1-NUTM2B, MRC1-PDGFRB, Cllorf95-YAP1, C11orf95-RELA, NUP98-KDM5A or CIC-FOXO4.

4. The method of claim 1, wherein the cleaving is done by an endonuclease selected from a CRISPR-associated protein, a zinc-finger nuclease (ZFN) and a transcription activator-like effector nuclease (TALEN).

5. The method of claim 4, wherein the CRISPR-associated protein is a Cas protein.

6. A method for treating a subject with an oncogenic gene fusion-associated cancer comprising administering an effective amount of an exogenous endonuclease that cleaves at least one neo splice site or cryptic exon of the oncogenic gene fusion of the subject thereby treating the subject.

7. The method of claim 6, wherein the oncogenic gene fusion-associated cancer is a leukemia, sarcoma, lymphoma, brain cancer, liver cancer, kidney cancer, lung cancer, prostate cancer, breast cancer, ovarian cancer, colon cancer, bladder cancer, salivary gland cancer, endocrine cancer, and gastric cancer.

8. The method of claim 6, wherein the cancer is a leukemia.

9. The method of claim 8, wherein the oncogenic gene fusion is MN1-PATZ1, CBFB-MYH11, C11orf95-NCOA2, TCF3-HLF, C11orf95-MAML2, BCOR-CCNB3, EWSR1-ATF1, MN1-CXXC5, TPM3-NTRK1, SPTBN1-ALK, FUS-FLI1, KAT6A-EP300, NUP98-BPTF, EP300-BCOR, CBFA2T3-GLIS2, Cllorf95-MAML2, ATXN1-NUTM2B, MRC1-PDGERB, Cllorf95-YAP1, C11orf95-RELA, NUP98-KDM5A or CIC-FOX04.

10. The method of claim 6, wherein the exogenous endonuclease is selected from a CRISPR-associated protein, a zinc-finger nuclease (ZFN) and a transcription activator-like effector nuclease (TALEN).

11. The method of claim 9, wherein the CRISPR-associated protein is a Cas protein.

12. A kit comprising at least one endonuclease and at least one guide RNA having a targeting domain complementary to a neo splice site or cryptic exon of an oncogenic gene fusion.

13. The kit of claim 12, wherein the at least one endonuclease is a Cas protein.

14. The kit of claim 12, wherein the oncogenic gene fusion is TCF3-HLF and the at least one guide RNA comprises SEQ ID NO:1-7.


SEQUENCE LISTING (77 sequences)

SEQ ID NO: 1 - DNA, 23 bp, synthetic construct
  source 1..23: mol_type = other DNA
atctcaggcgtgcccgactcngg

SEQ ID NO: 2 - DNA, 23 bp, synthetic construct
  source 1..23: mol_type = other DNA
ctgagatttctggtgcaggtngg

SEQ ID NO: 3 - DNA, 23 bp, synthetic construct
  source 1..23: mol_type = other DNA
gattctatcactcctaggccngg

SEQ ID NO: 4 - DNA, 23 bp, synthetic construct
  source 1..23: mol_type = other DNA
ctggggctgggaactccgtangg

SEQ ID NO: 5 - DNA, 23 bp, synthetic construct
  source 1..23: mol_type = other DNA
tgtatgactgtatcataacgngg

SEQ ID NO: 6 - DNA, 23 bp, synthetic construct
  source 1..23: mol_type = other DNA
caagtagatcgaggtggagangg

SEQ ID NO: 7 - DNA, 23 bp, synthetic construct
  source 1..23: mol_type = other DNA
ccagaattcttctgggtaggngg

SEQ ID NO: 8 - DNA, 101 bp, Homo sapiens
  source 1..101: mol_type = genomic DNA
gccctgtgccttccaccagcccaggaatcctgcctgctttccaggcagactttccaagtaccttgattctatcactcctaggccagggcatctcaccgcag

SEQ ID NO: 9 - DNA, 27 bp, Homo sapiens
  source 1..27: mol_type = genomic DNA
agggaccggagtcgggcacgcctgaga

SEQ ID NO: 10 - DNA, 101 bp, Homo sapiens
  source 1..101: mol_type = genomic DNA
tttctggtgcaggtgggtcattatttttaacagctgccaagtatccctttgtatgactgtatcataacgtggttgttaaatctcctatgcatagtttttcc

SEQ ID NO: 11 - DNA, 101 bp, Homo sapiens
  source 1..101: mol_type = genomic DNA
ccctgtgccttccaccagcccaggaatcctgcctgctttccaggcagactttccaagtaccttgattctatcactcctaggccagggcatctcaccgcagc

SEQ ID NO: 12 - DNA, 25 bp, Homo sapiens
  source 1..25: mol_type = genomic DNA
ttggtcccctctccacctcgatcta

SEQ ID NO: 13 - DNA, 101 bp, Homo sapiens
  source 1..101: mol_type = genomic DNA
cttgctcacccaggtattcttcaaagagcagcctcctccctcctacccagaagaattctggtaacatctattttgaaaatcgtttttttaccctgttgcat

SEQ ID NO: 14 - DNA, 101 bp, Homo sapiens
  source 1..101: mol_type = genomic DNA
tgcctcttcattcgcctgctcccagacgctgtgtgcctggcagcgctcagcactggggaaggggcgaggggtgcagcaggatgcctctgcctcaggggaga

SEQ ID NO: 15 - DNA, 101 bp, Homo sapiens
  source 1..101: mol_type = genomic DNA
acttgcagttgaggaaatgcagaaaatggaaagctgaagtcagcggatcactacctgttagagaaaggcttagcctggctcccaggttgtcttgcttccca

SEQ ID NO: 16 - DNA, 101 bp, Homo sapiens
  source 1..101: mol_type = genomic DNA
ggctgcggggaggacttgggatttggccatgagaaaggtggcagccgtggagggctgaggagggatgggacctgacccaggtgctcacagataccctctgg

SEQ ID NO: 17 - DNA, 101 bp, Homo sapiens
  source 1..101: mol_type = genomic DNA
cactgttgttaactggaggcttcaccactttgggccccctcaccaccatgacgtcattgactcgcctgactcctcccagcctctcccctgcctccagctcc

SEQ ID NO: 18 - DNA, 101 bp, Homo sapiens
  source 1..101: mol_type = genomic DNA
ggccctgtgccttccaccagcccaggaatcctgcctgctttccaggcagactttccaagtaccttgattctatcactcctaggccagggcatctcaccgca

SEQ ID NO: 19 - DNA, 12 bp, Homo sapiens
  source 1..12: mol_type = genomic DNA
gtctccaaaccc

SEQ ID NO: 20 - DNA, 101 bp, Homo sapiens
  source 1..101: mol_type = genomic DNA
gcttgctcacccaggtattcttcaaagagcagcctcctccctcctacccagaagaattctggtaacatctattttgaaaatcgtttttttaccctgttgca

SEQ ID NO: 21 - DNA, 101 bp, Homo sapiens
  source 1..101: mol_type = genomic DNA
ttagaacattattaaaactcgagtaatactactttccatttttctatgaatatttgtcttggttttataactataattgtcagtcatttgtgtgattttaa

SEQ ID NO: 22 - DNA, 101 bp, Homo sapiens
  source 1..101: mol_type = genomic DNA
ggccctgtccctggctcgggcccttgaagaggccttggaagccaaagaggaactcgagcggaccaacaaaatgctcaaagccgaaatggaagacctggtca

SEQ ID NO: 23 - DNA, 101 bp, Homo sapiens
  source 1..101: mol_type = genomic DNA
attacttattgtaactgtattattcctaaaagtataaggtcatgtttaactgaaattatataatattttgacccacaaattgattttattatattgctagc

SEQ ID NO: 24 - DNA, 101 bp, Homo sapiens
  source 1..101: mol_type = genomic DNA
gagcttcaggccgactctgccatcaaggggagggaggaagccatcaagcagctacgcaaactgcaggtgggtgacactaggagcttggggcatgggtggag

SEQ ID NO: 25 - DNA, 101 bp, Homo sapiens
  source 1..101: mol_type = genomic DNA
tatttagaaaaaaataaaatttgctttcagtattacacagaataatgaaaacagaagattctagaccttgctatctctattctctggcatataggctgttt

SEQ ID NO: 26 - DNA, 101 bp, Homo sapiens
  source 1..101: mol_type = genomic DNA
ccaagcgggccctggagacccagatggaggagatgaagacgcagctggaagagctggaggacgagctgcaagccacggaggacgccaaactgcggctggaa

SEQ ID NO: 27 - DNA, 101 bp, Homo sapiens
  source 1..101: mol_type = genomic DNA
tgtgattcagattatctttagagattcaaacatcatctaatctaacattattacagttgataaaactgagagtctccaaaaaattaattgacttgctctcc

SEQ ID NO: 28 - DNA, 101 bp, Homo sapiens
  source 1..101: mol_type = genomic DNA
ctcaacgtgtctacgaagctgcgccagctggaggaggagcggaacagcctgcaagaccagctggacgaggagatggaggccaagcagaacctggagcgcca

SEQ ID NO: 29 - DNA, 101 bp, Homo sapiens
  source 1..101: mol_type = genomic DNA
aaaattcccaactcccagcaatgctttgtctgattgttcatttgcagatgtcttagcgtgttaatttaaatgtcaaaggttttgaggtgtccagaaccacc

SEQ ID NO: 30 - DNA, 101 bp, Homo sapiens
  source 1..101: mol_type = genomic DNA
gctggtctttcatttgtcttgtttgtttttaagcaagaagaatccctttagaggaggaattaggaaagaaaaaaaagtcaaacagaaacagaaggagtgga

SEQ ID NO: 31 - DNA, 101 bp, Homo sapiens
  source 1..101: mol_type = genomic DNA
ccagaatgacgaccacgacgctgatgatgaggatgatggccacctggagtccacaaagaaaaaggagctagaggaacagcccacgagggaagatgtcaagg

SEQ ID NO: 32 - DNA, 101 bp, Homo sapiens
  source 1..101: mol_type = genomic DNA
tttttttttgagacggagtttagctcttgttgcccaggctggagtgcagtggtacgatctcggctcactgcaacttctccctcctggtttcaagcaactct

SEQ ID NO: 33 - DNA, 101 bp, Homo sapiens
  source 1..101: mol_type = genomic DNA
aacttttttgtatggatatgtagggcttggcgagtctaggtcaagcattccagccaaagaattgtgaaagatcacaacaatctgggaataacaaagattca

SEQ ID NO: 34 - DNA, 101 bp, Homo sapiens
  source 1..101: mol_type = genomic DNA
aagtccaagaaaaagaaaatgatctctactacctcaaaggaaactaagaaggacacaaagctttactgtatctgtaaaacgccttatgatgaatctaagtg

SEQ ID NO: 35 - DNA, 101 bp, Homo sapiens
  source 1..101: mol_type = genomic DNA
ggggcgcgctggccacgctcaaggtgagcaccatcaagcgccacatcctgcaggtgcaccccttctccatggacttcacgcctgaggagcgccagactatc

SEQ ID NO: 36 - DNA, 101 bp, Homo sapiens
  source 1..101: mol_type = genomic DNA
atccagaaatgtaatttattctcagtcttcactgaagagcatctggctcttgagctggaaatatggctctataagctttattgtatagctgagtttctctg

SEQ ID NO: 37 - DNA, 101 bp, Homo sapiens
  source 1..101: mol_type = genomic DNA
ctaccagccgcggtggcggggcgagtacctgatggactacgacggcagccggcgcggcctggtgtgtatggtgtgcgggggcgcgctggccacgctcaagg

SEQ ID NO: 38 - DNA, 101 bp, Homo sapiens
  source 1..101: mol_type = genomic DNA
tggtatgtaaattcaaaactagaataataggctacattatgtgctctcattgtctgaaaaataagttccctgaaaaaatccaggataccttaagtgatatt

SEQ ID NO: 39 - DNA, 101 bp, Homo sapiens
  source 1..101: mol_type = genomic DNA
tgagcaccatcaagcgccacatcctgcaggtgcaccccttctccatggacttcacgcctgaggagcgccagactatcctggaggcctacgaggaggcggcg

SEQ ID NO: 40 - DNA, 101 bp, Homo sapiens
  source 1..101: mol_type = genomic DNA
aggtgtggactaccacacctagcctaaatctagaactttctatgtatatatttacaaataatattttagatttttgttctctggttcaaattaacttctca

SEQ ID NO: 41 - DNA, 101 bp, Homo sapiens
  source 1..101: mol_type = genomic DNA
caggtgcaccccttctccatggacttcacgcctgaggagcgccagactatcctggaggcctacgaggaggcggcgctgcgctgctacggccacgagggctt

SEQ ID NO: 42 - DNA, 101 bp, Homo sapiens
  source 1..101: mol_type = genomic DNA
cgtccggcaacaaaggatgttttgtgctactactgaggtttgtgtgtgtgacttactttagaactctttctagaaaatgcgattactatttgcataggtct

SEQ ID NO: 43 - DNA, 101 bp, Homo sapiens
  source 1..101: mol_type = genomic DNA
gagcaccatcaagcgccacatcctgcaggtgcaccccttctccatggacttcacgcctgaggagcgccagactatcctggaggcctacgaggaggcggcgc

SEQ ID NO: 44 - DNA, 57 bp, Homo sapiens
  source 1..57: mol_type = genomic DNA
cggggcgggcggcccgaagccctcgtggggtaattaaaacgttattttcttttcttt

SEQ ID NO: 45 - DNA, 101 bp, Homo sapiens
  source 1..101: mol_type = genomic DNA
aaaaaatcagaataaacaatttggtcaagtaaaatatttccctccaagtagttaaggcaaagactgaaggaccatttgtaggaaatggagaatctttctat

SEQ ID NO: 46 - DNA, 101 bp, Homo sapiens
  source 1..101: mol_type = genomic DNA
ggcaactgaatctagcagtttggaggtcttagagcatttgtaataacatgctggctctctgtgaatgtcccagaaggaacatcttccatggaatggacttg

SEQ ID NO: 47 - DNA, 101 bp, Homo sapiens
  source 1..101: mol_type = genomic DNA
tccatgcggtctatgtggtaaggtgttcactgatgccaaccggctccggcagcacgaggcccagcacggtgtcaccagcctccagctgggctacatcgacc

SEQ ID NO: 48 - DNA, 101 bp, Homo sapiens
  source 1..101: mol_type = genomic DNA
actcttcgtgtgttctttgatcaagtcaggactattacttccattgcagggagactgaggcccagagagggaaagtgccttgtccaaagtcacacagctgg

SEQ ID NO: 49 - DNA, 101 bp, Homo sapiens
  source 1..101: mol_type = genomic DNA
ggcctgagggaggcaggcatccttccatgcggtctatgtggtaaggtgttcactgatgccaaccggctccggcagcacgaggcccagcacggtgtcaccag

SEQ ID NO: 50 - DNA, 101 bp, Homo sapiens
  source 1..101: mol_type = genomic DNA
gctcaagcattgctacgttcattccttgagataatttgtgcaaagtgggggaaataaccccctttcagattttctctctttctctctctctgtggcaggta

SEQ ID NO: 51 - DNA, 101 bp, Homo sapiens
  source 1..101: mol_type = genomic DNA
caagcctctttgcctgtgttacctggggtggaccgcttgcccatggtggctggacccctatccccccaactgctgacttccccattccccagtgtggcatc

SEQ ID NO: 52 - DNA, 101 bp, Homo sapiens
  source 1..101: mol_type = genomic DNA
ctgctttgcccatcagtctgtcctttcagagttgaagctgagctgctgtttgctgggcaggccatgcagcccacacgggggtcctcagaggccttgcaggg

SEQ ID NO: 53 - DNA, 10 bp, Homo sapiens
  source 1..10: mol_type = genomic DNA
acgtctctgg

SEQ ID NO: 54 - DNA, 101 bp, Homo sapiens
  source 1..101: mol_type = genomic DNA
gctgggctacatcgaccttcctcctccgaggctgggtgagaatgggctacccatctctgaagaccccgacggcccccgaaagaggagccggaccaggaagc

SEQ ID NO: 55 - DNA, 101 bp, Homo sapiens
  source 1..101: mol_type = genomic DNA
ccactgtcctacccgagtgaggcttgttacagacatcagggcccacctgactgtggtgggctacacgaggatgctcacatttcctccattagtcacctgat

SEQ ID NO: 56 - DNA, 101 bp, Homo sapiens
  source 1..101: mol_type = genomic DNA
gcgcgtggtgcaggagcatctcccgctgatgagcgaggcgggtgctggcctgcctgacatggaggctgtggcaggtgccgaagccctcaatggccagtccg

SEQ ID NO: 57 - DNA, 101 bp, Homo sapiens
  source 1..101: mol_type = genomic DNA
ccaggaaggtctagctcctgacacgttctatggtagagggaggagggttgatgcttgctcaggttacttgggaacatctcttccccagtatgccttccaac

SEQ ID NO: 58 - DNA, 101 bp, Homo sapiens
  source 1..101: mol_type = genomic DNA
tctcggtggctgtgggcctggccgtctttgcctgcctcttcctttctacgctgctccttgtgctcaacaaatgtggacggagaaacaagtttgggatcaac

SEQ ID NO: 59 - DNA, 101 bp, Homo sapiens
  source 1..101: mol_type = genomic DNA
ccgccactttgctggcacctgctctaaacatctggtctctgctgcttggctctcaggagcaaaggtataaggacgtggccaatgctaggttattagcttag

SEQ ID NO: 60 - DNA, 101 bp, Homo sapiens
  source 1..101: mol_type = genomic DNA
atgcactagcccactcttccccaaaccagccctccaccaccctccaggcagagagataggaaaatcggtttctgagtatatttctgttcagcctgtgagcc

SEQ ID NO: 61 - DNA, 101 bp, Homo sapiens
  source 1..101: mol_type = genomic DNA
catatgaaaatatctcttctttatataagagaaattactccagtcagaaggacttagaaacatgtttttttccttttaaacttttaagtcagtttttatga

SEQ ID NO: 62 - DNA, 101 bp, Homo sapiens
  source 1..101: mol_type = genomic DNA
cttttagtaattcagtacctgtttgagctagtctgtgctttatagtgtggagacaacttaactttccagggattctcagcagctgactggtagcttgccag

SEQ ID NO: 63 - DNA, 101 bp, Homo sapiens
  source 1..101: mol_type = genomic DNA
cttggtgatataactttgttttgtttacagagtacctgctcgggccaggtaaatgctattggatgtaatccagtagtgtgtaatataaattcaaaccatat

SEQ ID NO: 64 - DNA, 101 bp, Homo sapiens
  source 1..101: mol_type = genomic DNA
gtgtggccaccacaccagctttttttttttttttttcgtatttttgtagagacatggtttcactatgttgggcaggctggtctcgaactcctgacctgaag

SEQ ID NO: 65 - DNA, 101 bp, Homo sapiens
  source 1..101: mol_type = genomic DNA
taaatagcattttttaaaaaacagaatgaacttcaaaattaaagttgatttttaacttccatattagcaaatactcttcactactgaaagacagtactatt

SEQ ID NO: 66 - DNA, 101 bp, Homo sapiens
  source 1..101: mol_type = genomic DNA
caaggtacaactattcttcagtatgcacagacctctgatggacagcagatacttgtgcccagcaatcaggtggtcgtacaaagtaagtatgctttctgtct

SEQ ID NO: 67 - DNA, 592 bp, Homo sapiens
  source 1..592: mol_type = other DNA
gacgaggaggaggagccagaggaggaggaggaggagtggggcgacgttccgctgtcccctggagctcccttggagcggcccgccgaagaagaggaggacgaagaggacggccaggagcctgggggactcgccttgccgccgccgcctcctcccccgcctccgcccccgccccgcagccgggagcagcggcggaactaccagccgcggtggcggggcgagtacctgatggactacgacggcagccggcgcggcctggtgtgtatggtgtgcgggggcgcgctggccacgctcaaggtgagcaccatcaagcgccacatcctgcaggtgcaccccttctccatggacttcacgcctgaggagcgccagactatcctggaggcctacgaggaggcggcgctgcgctgcgacctggaggcgctcttcaacgccgtcatgaaccccaagacggccaacgtgccccagaccgtgcccatgaggctccggaagctgcccgactccttcttcaagccgccggagcccaaatcccactcccgacaggccagttgtatagtctcctgtcggagaccaaagggttttggaactcagaaaaaat

SEQ ID NO: 68 - DNA, 496 bp, Homo sapiens
  source 1..496: mol_type = other DNA
ccgggagcagcggcggaactaccagccgcggtggcggggcgagtacctgatggactacgacggcagccggcgcggcctggtgtgtatggtgtgcgggggcgcgctggccacgctcaaggtgagcaccatcaagcgccacatcctgcaggtgcaccccttctccatggacttcacgcctgaggagcgccagactatcctggaggcctacgaggaggcggcaggagctggcaaacacaccaaggccaccgccactgctgccaccactacagcccctccaccgccccctgctgcccctcctgcggcctcccaagcagcagcaacagcagccccaccgcccccaccagactatcaccatcaccaccagcagcacctgctgaacagtagcaataatggtggcagtggtgggataaacggagagcagcagccgcccgcttcaaccccaggggaccagaggaactcagccctgattgcggatattccttaactgataagaagc

SEQ ID NO: 69 - DNA, 507 bp, Homo sapiens
  source 1..507: mol_type = other DNA
gatcgactccagcaccgtagagaggattgaagacagccatagcccgggcgtggccgtgatacagttcgccgtcggggagcaccgagcccaggtcagcgttgaagttttggtagagtatcctttttttgtgtttggacagggctggtcatcctgctgtccggagagaaccagccagctctttgatttgccgtgttccaaactctcagttggggatgtctgcatctcgcttaccctcaagaacctgaagaacggctctgttaaaaagggccagcccgtggatcccagcaaggccggccccaaggccccgactgcctgcctgccaccacccaggccccagaggccagtgaccaaggcccgccggccaccaccccggccccaccggcgagcagagaccaaggcccgcctgccaccacccaggccccagagaccagcagagaccaaggtccctgaggagatccccccagaagtggtgcaggagtatgtggacatcatggaggagctgctagg

SEQ ID NO: 70 - DNA, 521 bp, Homo sapiens
  source 1..521: mol_type = other DNA
cctacaaaggatatatttgtaaaagaccaaaaattattgatgctaaacctactcatgaattacttacaacaaaagctgacacaaggaagatggacccttctaaaccgtcttccaacgtggccggagtagtcatcattgtgatcctcctgattttaacgggtgctggccttgccgcctatttcttttataagaaaagacgtgtgcacctacctcaagagggcgcctttgaaaacactctgtattttgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccac

SEQ ID NO: 71 - DNA, 385 bp, Homo sapiens
  source 1..385: mol_type = other DNA
gtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgatttatgtctacccgctgcttactgtgagcgtgcaatgatgcgcttctcagagttggagatgaaagaaagagaaggtggccacccagcaaccaaagactccgagatgtgcc

SEQ ID NO: 72 - DNA, 423 bp, Homo sapiens
  source 1..423: mol_type = other DNA
tggaactgcgggcggaaagccagtgagacgtgcagcggctgcaacgcggcacgctactgcgggtccttctgccagcatcgggactgggagaagcatcaccacgtgtgtggccagagcctgcagggccccacagccgtggtggccgacccggtgcctggaccgcccgaagccgcccacagcctgggcccctccctgcctgtgggtgctgccagcctggtggatgacagccccacacctggctctccaggctccccgccctcaggcttcctgctgaactccaagttccccgagaaggtggagggacgcttttcagcagcccctctcgtggacctcagcctgtcaccaccatctgggctggactcccccaatggcagcagctcgctgtcccccgagcgccagggcaacggggacctgcctccagtg

SEQ ID NO: 73 - DNA, 709 bp, Homo sapiens
  source 1..709: mol_type = other DNA
ggcgcctggagaggaggctgaaggagtccctgcagaactggttccgggccgagtgtctcatggactatgacccgcgggggaaccggctggtgtgcatggcctgtggccgggcactgcccagcctgcacctggacgacatccgtgcccacgtgctggaggtgcaccctggctccctggggctcagcggcccccagcgcagtgccctgctgcaggcctgggggggccagcccgaggcgctgtctgagctcacccagtccccaccaggcgatgacctcgccccccaggacctgaccggaaagagccgggactcggcctccgctgctggagccccctcctctcaggatccctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaa

SEQ ID NO: 74 - DNA, 870 bp, Homo sapiens
  source 1..870: mol_type = other DNA
aaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacctgtgttctccggctctccgcccatgaagagtctttcatccaccagtgcaggcggcaaaaagcaggctcagccaagctgcgcaccagcctccaggccgcctgccaaacagcagaaaattaaagaaaaccagaagacagatgtgctgtgtgcagacgaagaagaggattgccaggctgcctccctgctgcagaaatacaccgacaacagcgagaagccatccgggaagagactgtgcaaaaccaaacacttgatccctcaggagtccaggcggggattgccactgacaggggaatactacgtggagaatgccgatggcaaggtgactgtccggagattcagaaagcggccggagcccagttcggactatgatctgtcaccagccaagcaggagccaaagcccttcgaccgcttgcagcaactgctaccagcctcccagtccacacagctgccatgctcaagttcccctcagg

SEQ ID NO: 75 - DNA, 175 bp, Homo sapiens
  source 1..175: mol_type = other DNA
cgcgaggagctcaaccactgggcgcggcgctacagcgacgccgaggacacaaagaagggccccgctcccgccgcggcccggccccgcagcagctccgccggtcccgagtgcctctcgccagacctgcccctgcccaagcagctggtgtgtcgctgggccaagtgtaaccagctct

SEQ ID NO: 76 - DNA, 219 bp, Homo sapiens
  source 1..219: mol_type = other DNA
atttggaacagctcttggtgctggacaggcatctttgtttgggaacaaccaacctaagattggagggcctcttggtacaggagcctttggggcccctggatttaatactacgacagccactttgggctttggagccccccaggccccagtagaaaaggtagagcaactttttggagaaggaaaacagaagtccaaggagttaaagaaaatggacaaacc

SEQ ID NO: 77 - DNA, 455 bp, Homo sapiens
  source 1..455: mol_type = other DNA
ctgcggcgcaccctggaccagcgccgggccctggtcatgcagctctttcaggaccatggcttcttcccgtcagcccaggccacagccgccttccaggcccgctatgcagacatctttccctccaaggtttgtctgcagttgaagatccgtgaggtgcgccagaagatcatgcaggctgccactcccacggagcagccccctggagctgaggctcctctccctgtaccgccccccactggcaccgctgctgcccctgcccccactcccagccccgcagggggccctgaccccacctcacccagctcggactctggcacggcccaggctgccccgccactgcctccacccccagagtcggggcctggacagcctggctgggaggttaccggccccttacacacctacagcagctcccttttcagcccagcagaggggcccctgtcagcaggagaagg